(19) United States
(12) Patent Application Publication
(10) Pub. No.: US 2014/0127761 A1
(43) Pub. Date: May 8, 2014

(54) Method for Producing Fatty Acid

(71) Applicant: AJINOMOTO CO., INC., Tokyo (JP)

(72) Inventor: Shigeo Suzuki, Kanagawa (JP)

(73) Assignee: AJINOMOTO CO., INC., Tokyo (JP)

(21) Appl. No.: 14/154,501

(22) Filed: Jan. 14, 2014

Related U.S. Application Data
(63) Continuation of application No. PCT/JP2012/067996, filed on Jul. 13, 2012.

(30) Foreign Application Priority Data
Jul. 14, 2011 (JP) 2011-155836

(51) Int. Cl. C12P 7/64

(52) U.S. Cl. CPC C12P 7/6436
USPC 435/106; 435/134

(57) ABSTRACT

A method for producing a fatty acid, which comprises adding an organic solvent to a culture of an alga obtained by culturing the alga in a culture medium, and stirring the culture medium to allow a transesterification or hydrolysis reaction of a lipid, and collecting a fatty acid ester or a fatty acid from the reaction mixture.

This application is a Continuation of, and claims priority under 35 U.S.C. §120 to, International Application No. PCT/JP2012/067996, filed Jul. 13, 2012, and claims priority therethrough under 35 U.S.C. §119 to Japanese Patent Application No. 2011-155836, filed Jul. 14, 2011, the entireties of which are incorporated by reference herein.

### FIELD OF THE INVENTION

The present invention relates to a method for producing fatty acids using algae. Fatty acids are used in various fields, such as those of food additives, chemicals, cosmetics, and drugs.

### BRIEF DESCRIPTION OF THE RELATED ART

Fatty acids include those obtained by adding an alcohol and a catalyst to fats or oils derived from animals, plants, fishes, and waste oils, allowing transesterification to obtain fatty acid esters, and hydrolyzing the fatty acid esters and fats or oils, and so forth. As the methods for the transesterification, there are known methods of using a catalyst consisting of an acid, an alkali, a metal, a lipase, or the like. Examples include, for example, the methods disclosed in U. Schuchardt et al., 1998, J. Brazilian Chemical Society, 9 (3), 199-210, H. Fukuda, A. Kondo, H. Noda, 2001, J. Bioeng, 92, 405-416, T. Samukawa et al., 2000, J. Biosci. and Bioeng., 90, 180-183, and L. A. Nelson et al., 1996, J. Am. Oil Soc. Chem., 73, 1191-1195. Other than the methods utilizing a catalyst, the supercritical method is used. Examples include, for example, the methods disclosed in S. Saka, D. Kusdiana, 2001, Fuel, 80, 225-231, D. Kusdiana, S. Saka, 2001, J. Chem. Eng. Japan, 34, 383-387, and D. Kusdiana, S. Saka, 2001, Fuel, 80, 693-698. Also for hydrolysis of a fatty acid, there are known methods of using a catalyst consisting of an acid, an alkali, a lipase, or the like, methods of using a high temperature and high pressure treatment, and so forth as in the case of the aforementioned transesterification. Examples include, for example, the methods disclosed in Japanese Patent Laid-open (Kokai) No. 2010-106107 and Japanese Patent Laid-open No. 2003-113395.

In the industrial production of fatty acids based on transesterification or hydrolysis, fish oils, animal oils, vegetable oils, waste oils, and so forth are used as fats and oils. Fats and oils derived from higher plants, such as soybean and palm, are frequently used as a source of fat or oil in methods for producing fatty acid ester by transesterification. These fats and oils are easily industrially obtainable from seeds by compression or solvent extraction. On the other hand, fats and oils which originate from microalgae are present at a concentration comparable to that of soybean or palm seeds in terms of dry weight, but dry alga body weight in culture fluid of algae is less than 1% of the culture fluid. In addition, the process of separating alga bodies, dehydrating them, disrupting cells, extracting fats and oils, and purifying them is complicated and difficult. It is possible to produce a fatty acid ester or fatty acid from fat or oil purified from algae by using an acid, an alkali, or a lipase (International Patent Publication WO2010/000416 and International Patent Publication WO2009/093703, N. Nagle, P. Lemke, 1990, Applied Biochem. and Biotech., 24, 355-361 and A. Robles Medina et al., 1999, J. Biotech., 70, 379-391). Further, in the methods of U.S. Patent Published Application No. 2008/0241902, China Patent Published Application No. 101580857, and U.S. Patent Published Application No. 2009/0158638, an alcohol is added to a microalga, and transesterification of fats and oils are directly induced within cells, but all the methods require an acid or an alkaline catalyst for the transesterification.

It is known that Synechocystis algae, which are typical recombinant producible algae, can produce a large amount of fatty acids, since they express acetyl-CoA carboxylase and thioesterase (X. Liu et al., 2009, PNAS, 24, 1-6), and they can produce triglycerides, since they express diacylglycerol acetyltransferase (U.S. Patent Published Application No. 2010/0081178). Therefore, it is easy to produce a fatty acid ester from fats and oils of Synechocystis algae by using a catalyst such as an acid, an alkali, a lipase, or the like. Further, it is also known that a fatty acid ester or a fatty acid can be produced by expressing a catalytic enzyme by a genetic recombination technique without adding a exogenous catalyst such as an acid, an alkali, or a lipase. For example, a microalga is made to express pyruvate decarboxylase and alcohol dehydrogenase and thereby made to produce ethanol, and the microalga is further made to express ethanol acetyltransferase or an esterification enzyme to produce a fatty acid ester within the cells thereof (International Patent Publication WO2010/011754 and China Patent Published Application No. 2011101892092). Furthermore, a Rhodococcus bacterium is made to express a lipase to produce a fatty acid from intracellular lipids (International Patent Publication WO2011/008058). These methods for producing a fatty acid ester or fatty acid are based on genetic recombination for expressing a gene for a catalyst of the esterification or hydrolysis reaction, and methods for producing a fatty acid ester or fatty acid from fat or oil within alga cells without using genetic recombination techniques has not been previously reported.

Algae generally use lipases for decomposition of lipids of cell membranes or fats and oils (K. Hoehne-Reitan et al., 2007, Aqua. Nutri., 13, 45-49). For green algae (Chlorophyceae algae), it has been confirmed that intracellular fats and oils can be decomposed into fatty acids by a treatment at a mid-temperature under a weakly acidic condition (International Patent Publication WO2011/013707). Further, an increase in the lipase activity and decomposition of fats and oils into fatty acids induced by silica starvation have been confirmed also in diatoms (N. Nagle et al., 1989, Energy from Biomass and Wastes, 12, 1107-1115). However, production of a fatty acid ester or fatty acid from fats and oils in alga cells by adding an alcohol or an organic solvent other than alcohol to the cells has not been previously reported.

### SUMMARY OF THE INVENTION

The present invention provides a more efficient method for producing a fatty acid, especially at a lower cost without the addition of a catalyst. This is in contrast to conventional fatty acid production methods which utilize fats and oils mainly derived from animals, plants, fishes and waste fluids as substrates and also employing an acid or alkaline catalyst.

It has been found that by adding only an alcohol or an organic solvent other than alcohol to a culture of an alga, fatty acid esters or fatty acids are efficiently produced in the alga cells without adding an acid or an alkali.

The present invention thus provides the following.

It is an aspect of the present invention to provide a method for producing a fatty acid, which comprises culturing an alga in a culture medium, adding an organic solvent to the culture medium to obtain a mixture, stirring the mixture to allow a transesterification or hydrolysis reaction of a lipid, and collecting a fatty acid ester or a fatty acid from the mixture.

It is a further aspect of the present invention to provide the method as described above, wherein the organic solvent is ethanol, and a fatty acid ester is collected.

It is a further aspect of the present invention to provide the method as described above, wherein the organic solvent is acetone, chloroform, ethyl acetate, methyl acetate, hexane, benzene, toluene, dichloromethane, acetonitrile, dimethyl ether, and diethyl ether; and a fatty acid is collected.

It is a further aspect of the present invention to provide the method as described above, wherein concentration of the organic solvent in the mixture is 5% or higher.

It is a further aspect of the present invention to provide the method as described above, wherein concentration of the organic solvent in the mixture is 65% or lower.

It is a further aspect of the present invention to provide the method as described above, wherein the organic solvent is a lower alcohol having 5 or less carbon atoms.

It is a further aspect of the present invention to provide the method as described above, wherein the organic solvent is a higher alcohol having 6 or more carbon atoms.

It is a further aspect of the present invention to provide the method as described above, wherein the reaction is allowed at a temperature of 10° C. or higher.

It is a further aspect of the present invention to provide the method as described above, wherein the reaction is allowed at a temperature of 60° C. or lower.

It is a further aspect of the present invention to provide the method as described above, wherein the reaction is allowed at a weakly acidic to weakly alkaline pH.

It is a further aspect of the present invention to provide the method as described above, wherein the collecting the fatty acid ester or fatty acid comprises treating the reaction product with an organic solvent.

It is a further aspect of the present invention to provide the method as described above, wherein the alga is a microalga.

It is a further aspect of the present invention to provide the method as described above, wherein the microalga belongs to the phylum Chlorophyta.

It is a further aspect of the present invention to provide the method as described above, wherein the alga is a microalga belonging to the class Chlorophyceae, Trebouxiophyceae, or Prasinophyceae.

It is a further aspect of the present invention to provide the method as described above, wherein the alga is a microalga belonging to the class Chlorophyceae.

It is a further aspect of the present invention to provide the method as described above, wherein the alga is a freshwater microalga belonging to the class Chlorophyceae.

It is a further aspect of the present invention to provide the method as described above, wherein the alga is a marine microalga belonging to the class Chlorophyceae, and is a microalga that accumulates fats and oils as storage substances.

It is a further aspect of the present invention to provide a method for producing an L-amino acid, which comprises producing a fatty acid by the method as described above, culturing a bacterium having an L-amino acid-producing ability in a medium containing the fatty acid to produce and accumulate an L-amino acid in culture, and collecting the L-amino acid from the culture.

It is a further aspect of the present invention to provide the method as described above, wherein the bacterium is a bacterium belonging to the family Enterobacteriaceae or a coryneform bacterium.

It is a further aspect of the present invention to provide the method as described above, wherein the bacterium belonging to the family Enterobacteriaceae is Escherichia coli.

Fatty acid esters or fatty acids can be efficiently produced according to the present invention.

### BRIEF DESCRIPTION OF THE DRAWINGS

FIG. 1 shows results of examination of temperature conditions for the reaction of alga culture after addition of an alcohol.

FIG. 2 shows results of examination of methanol concentration for the reaction of alga culture after addition of an alcohol.

FIG. 3 shows results of examination of pH condition for the reaction of alga culture after addition of an alcohol.

FIG. 4 shows results of examination of reaction time for the reaction of alga culture after addition of an alcohol.

FIG. 5 shows results of examination of type of alcohol to be added for the reaction of alga culture after addition of an alcohol (photograph).

FIG. 6 shows results of identification of fatty acid methyl esters produced in the reaction of alga culture after addition of an alcohol.

FIG. 7 shows results of the reaction performed with cultures of Chlorophyceae algae after addition of an alcohol.

FIG. 8 shows confirmation of fatty acid production in the reaction of alga culture after addition of an organic solvent other than alcohol.

FIG. 9 shows results of examination of type of organic solvent to be added for the reaction of alga culture after addition of an organic solvent (photograph).

### DESCRIPTION OF THE PREFERRED EMBODIMENTS

Hereafter, the present invention will be explained in detail.

<1> Algae and culture method therefor

As the alga, any algae can be used. However, microalgae which accumulate fats and oils in alga bodies are preferred particular example.

“Algae” can refer to all organisms performing oxygen generating type photosynthesis except for Bryophyta, Pteridophyta and Spermatophyta, which live mainly on the ground. Algae can include various unicellular organisms and multicellular organisms such as cyanobacteria (blue-green algae), which are prokaryotes, as well as those classified into the phylum Glaucophyta, Rhodophyta (red algae), Chlorophyta, Cryptophyta (crypt algae), Haptophyta (haptophytes), Heterokontophyta, Dinophyta (dinoflagellates), Euglenophyta, or Chlorarachniophyta, which are eukaryotes. “Microalgae” can refer to algae having a microscopic structure except for multicellular marine algae (Biodiversity Series (3) Diversity and Pedigree of Algae, edited by Mitsuo Chihara, Shokabo Publishing Co., Ltd. (1999)).

It is known that some plants, including algae, can accumulate fats and oils as storage substances (Chisti Y., 2007, Biotechnol. Adv., 25:294-306). As such algae, those belonging to the phylum Chlorophyta or Heterokontophyta are well known. Examples of the algae belonging to the phylum Chlorophyta include those belonging to the class Chlorophyceae, and examples of algae belonging to the class Chlorophyceae include Chlorella minutissima (Bhatnagar A., 2010, Appl. Biochem. Biotechnol., 161:523-36), Scenedesmus obliquus (Shovon, M. et al., 2009, Appl. Microbiol. Biotechnol., 84:281-91), Neochloris oleoabundans (Tornabene, T. G. et al., 1983, Enzyme and Microb. Technol., 5:435-440), Nannochloris sp. (Takagi, M. et al., 2000, Appl. Microbiol. Biotechnol., 54:112-117) and so forth. In the phylum Heterokontophyta, the classes Chrysophyceae, Dictyochophyceae, Pelagophyceae, Rhaphidophyceae, Bacillariophyceae, Phaeophyceae, Xanthophyceae, and Eustigmatophyceae are classified. Examples of frequently used algae belonging to the class Bacillariophyceae include Thalassiosira pseudonana (Tonon, T. et al., 2002, Phytochemistry, 61:15-24). Specific examples of Chlorella minutissima include the Chlorella minutissima UTEX 2314 strain, specific examples of Scenedesmus obliquus include the Scenedesmus obliquus UTEX393 strain, specific examples of Neochloris oleoabundans include the Neochloris oleoabundans UTEX 1185 strain, specific examples of Nannochloris sp. include the Nannochloris sp. UTEX LB 1999 strain, and specific examples of Thalassiosira pseudonana include the Thalassiosira pseudonana UTEX LB FD2 strain. These strains can be obtained from the University of Texas at Austin, The Culture Collection of Algae (UTEX), University Station A6700, Austin, Tex. 78712-0183, USA. Further, as algae that produce EPA and DHA, which are highly functional fatty acids, those belonging to the phylum Chlorophyta, Heterokontophyta, Rhodophyta, or Haptophyta are known well. Examples of algae belonging to the phylum Chlorophyta include algae belonging to the class Chlorophyceae, Prasinophyceae, or Trebouxiophyceae, and examples of well-known algae belonging to the class Chlorophyceae include Chlorella minutissima (Rema V. et al., 1998, JAOCS. 75:393-397). Examples of algae belonging to the phylum Heterokontophyta include algae belonging to the class Bacillariophyceae or Eustigmatophyceae, examples of algae belonging to the class Bacillariophyceae and which are frequently used include Thalassiosira pseudonana (Tonon, T. et al., 2002, Phytochemistry 61:15-24), and examples of algae belonging to the class Eustigmatophyceae include Nannochloropsis oculata. Freshwater Chlorophyceae algae such as those of the genera Chlorella and Scenedesmus are particular examples.

There is much information about culture of microalgae, and those of the genus Chlorella or Arthrospira (Spirulina), Dunaliella salina and so forth are industrially cultured in a large scale for use as food (Spolaore, P. et al., 2006, J. Biosci. Bioeng., 101:87-96). For Chlamydomonas reinhardtii, for example, the 0.3×HSM medium (Oyama Y. et al., 2006, Planta, 224:646-654) can be used, and for Chlorella kessleri, the 0.2× Gamborg's medium (Izumo A. et al., 2007, Plant Science, 172:1138-1147) and so forth can be used. For Chlorella vulgaris, the BG-11 medium, the M8 medium (Ramkumar, K. M. et al., 1998, Biotech. Bioeng., 59:605-611) and so forth can be used. Neochloris oleoabundans and Nannochloris sp. can be cultured by using the modified NORO medium (Yamaberi, K. et al., 1998, J. Mar. Biotechnol., 6:44-48; Takagi, M. et al., 2000, Appl. Microbiol. Biotechnol., 54:112-117), the Bold's basal medium (Tornabene, T. G. et al., 1983, Enzyme and Microb. Technol., 5:435-440; Archibald, P. A. and Bold, H. C., 1970, Phytomorphology, 20:383-389), or the Daigo's IMK medium (Ota M. et al., 2009, Bioresource Technology, 100:5237-5242). For Thalassiosira pseudonana as an alga belonging to the class Bacillariophyceae, the F/2 medium (Lie, C.-P. and Lin, L.-P., 2001, Bot. Bull. Acad. Sin., 42:207-214) and so forth can be preferably used. Further, a photobioreactor can also be used for culture of microalgae (WO2003/094598).

The culture is usually performed by adding 1 to 50% of precultured cell suspension based on the volume of main culture. Initial pH can be around neutral, i.e., 6 to 9, and pH adjustment is not usually necessary during culture in many cases. However, pH may be adjusted if needed. The culture temperature can be 25 to 35° C., and in particular, a temperature around 28° C. is generally frequently used. However, the culture temperature may be a temperature suitable for the chosen alga. Air is usually blown into the culture medium, at an aeration rate and aeration volume per unit volume of culture medium per minute of 0.1 to 2 vvm (volume per volume per minute). Further, CO₂ may also be blown into the medium in order to promote growth, and can be added at about 0.5 to 5% of the aeration rate. Although optimum illumination intensity of light also differs depending on type of microalgae, an illumination intensity of about 1,000 to 30,000 luxes can be usually used. As the light source, it is common to use a white fluorescent lamp indoors, but the light source is not limited to this. It is also possible to perform the culture outdoors with sunlight. The culture medium may be stirred at an appropriate intensity, or circulated, if needed. Further, it is known that algae accumulate fats and oils in alga bodies when nitrogen source is depleted (Thompson G. A. Jr., 1996, Biochim. Biophys. Acta, 1302:17-45), and a medium of a limited nitrogen source concentration can also be used for the main culture.

The culture of an alga can include a culture fluid or medium containing alga bodies, and alga bodies collected from the culture fluid or medium.

Alga bodies can be collected from the culture fluid or medium by typical methods, such as centrifugation, filtration, gravitational precipitation using a flocculant, or the like (Grima, E. M. et al., 2003, Biotechnol. Advances, 20:491-515).

Although an alcohol or an organic solvent other than alcohol can be directly added to the culture fluid or medium, the microalga can also be concentrated by centrifugation or the like, before the addition. The concentration operation of alga bodies can include removing the solvent component to obtain a concentration of 25 g/L or higher, or 250 g/L or higher, as a concentration of microalga in terms of dry weight in the solution (including suspending alga bodies separated from a medium by centrifugation or the like in a liquid to obtain a desired concentration), and a process of precipitating alga bodies, separating them from a medium and using them.

<2> Method for Reaction Utilizing Culture of Alga and Reaction Product

A mixture obtained by adding an organic solvent to a culture of an alga is stirred to allow a transesterification or hydrolysis reaction of a lipid, and a fatty acid ester or a fatty acid is collected from the reaction product. The fatty acid ester and fatty acid may be collectively referred to as “fatty acid”.

When an alcohol is added to a culture of an alga, generation of a fatty acid ester is mainly induced by transesterification of a lipid and an alcohol, and when an organic solvent other than alcohol is added to a culture of an alga, generation of a fatty acid is mainly induced by hydrolysis of a lipid.

The reaction product can mean a reaction mixture obtained by adding an alcohol or an organic solvent other than alcohol to a culture of an alga to obtain a mixture, and stirring the mixture to allow a transesterification or hydrolysis reaction of a lipid. The reaction product or reaction mixture may be further subjected to extraction, fractionation, and/or another treatment, so long as the subsequent collection of a fatty acid ester or fatty acid is not inhibited. By-products other than fatty acid ester are produced in the reaction product, such as glycerol which may be utilized for production of L-amino acids utilizing bacteria having an L-amino acid-producing ability or production of chemical products.

The reaction using the mixture obtained by adding an alcohol or an organic solvent other than alcohol may be allowed at a temperature sufficient for increasing a fatty acid ester or fatty acid in the reaction product. As for the minimum temperature, the reaction is usually performed at a temperature of 10° C. or higher, 15° C. or higher, or 20° C. or higher, and as for the maximum temperature, the reaction is usually performed at a temperature of 60° C. or lower, 50° C. or lower, or 40° C. or lower.

In the reaction, a culture obtained by the aforementioned culture method of algae may be used as it is, or it may be concentrated before use as described above. For example, the mixture may be once centrifuged, and then precipitated alga bodies may be used as the reaction product.

Further, pH of the mixture may be adjusted before the addition of an alcohol or an organic solvent other than alcohol so that pH becomes an acidic to weakly alkaline pH, which is usually 2.0 to 11.0, 3.0 to 10.5, or 3.5 to 9.0, during the reaction, or it may also be adjusted so that pH is a weakly acidic to weakly alkaline pH during the reaction.

An alcohol or other organic solvent can be added before the reaction at a concentration that permits the reaction, such as at least 5 volume % or higher, 15 volume % or higher, or 25 volume % or higher. As for the maximum concentration, the reaction can occur at a concentration of usually 65 volume % or lower, 55 volume % or lower, or 45 volume % or lower.

As the alcohol to be added, a lower alcohol having 5 or less carbon atoms such as methanol, ethanol, propanol, isopropanol, butanol, pentanol and ethylene glycol, or a higher alcohol having 6 or more carbon atoms such as hexanol, heptanol, octanol, nonanol, decanol, undecanol, dodecanol, tridecanol and tetradecanol may be used.

As the organic solvent other than alcohol, acetone, chloroform, ethyl acetate, methyl acetate, hexane, benzene, toluene, dichloromethane, acetonitrile, dimethyl ether, diethyl ether, and so forth may be used.

The reaction using the mixture obtained by adding an alcohol or an organic solvent other than alcohol is usually performed for at least 10 minutes or longer, 20 minutes or longer, or 30 minutes or longer at a minimum. The maximum reaction time is typically 15 hours or shorter, 10 hours or shorter, or 5 hours or shorter.

The means for the stirring is not limited so long as the effect of the aforementioned reaction can be obtained, and stirring by rotation shaking, vortex stirring with a vortex mixer, and so forth can be used. When water solubility of the alcohol or the organic solvent other than alcohol is low, a strong stirring means such as vortex stirring can be used in order to maintain a sufficiently mixed state.

As the method for collecting the fatty acid ester or fatty acid from the reaction product obtained after the reaction, common methods for extracting fats and oils from algae can be used, and examples include, for example, treatment with organic solvent, ultrasonication, beads mill treatment, acid treatment, alkali treatment, enzyme treatment, hydrothermal treatment, supercritical treatment, microwave treatment, electromagnetic field treatment, compression treatment, and so forth, and the Bligh-Dyer method (rapid method of total lipid collection and purification, Can. J. Biochem. Physiol., 37:911-917) is a particular example. A method of eluting the fatty acid ester or fatty acid out of the cells, and collecting the fatty acid ester or fatty acid from the effluent is a particular example.

It is considered that the reason why addition of a catalyst is not required is that transesterification of fats, oils, ceramides, phospholipids, phospholipids, and glycolipids with the alcohol added from the outside or hydrolysis of the same is induced by non-recombinant lipases in cells of alga.

<3> Bacterium

The fatty acid obtained by the method described above can be used as a carbon source for L-amino acid fermentation. A bacterium having an L-amino acid-producing ability can be used for L-amino acid production.

The bacterium is not particularly limited, so long as a bacterium which can efficiently produce an L-amino acid from a fatty acid produced by microalgae is chosen. Examples of the bacterium include, for example, bacteria belonging to the family Enterobacteriaceae such as those of the genus Escherichia, Pantoea, Enterobacter, or the like, and so-called coryneform bacteria such as those belonging to the genus Brevibacterium, Corynebacterium, Microbacterium, or the like, but the bacterium is not limited to these. Particular examples of the bacteria belonging to the family Enterobacteriaceae include Escherichia coli.

The L-amino acid-producing bacterium may be modified to increase an ability to utilize hydrolysate of fat or oil or fatty acid. Examples of such modification include, for example, deletion of the gene coding for the transcription factor FadR having a DNA-binding ability for controlling the fatty acid metabolism observed in Enterobacteriaceae bacteria (DiRusso, C. C. et al., 1992, J. Biol. Chem., 267:8685-8691; DiRusso, C. C. et al., 1993, Mol. Microbiol., 7:311-322). Specifically, the fadR gene of Escherichia coli is a gene located at the nucleotide numbers 1,234,161 to 1,234,880 of the genome sequence of Escherichia coli MG1655 strain registered with GenBank Accession No. U00096, and coding for the protein registered with GenBank accession No. AAC74271.

In order to enhance the ability to assimilate a hydrolysate of fats and oils or a fatty acid, expression amounts of one or more kinds of genes selected from fadA, fadB, fadI, fadJ, fadL, fadE and fadD may be enhanced.

The “fadL gene” can mean a gene encoding a transporter of the outer membrane having an ability to take up a long chain fatty acid, which is found in the Enterobacteriaceae bacteria (Kumar, G. B. and Black, P. N., 1993, J. Biol. Chem., 268:15469-15476; Stenberg, F. et al., 2005, J. Biol. Chem., 280:34409-34419). Specific examples of a gene encoding FadL include the gene located at the nucleotide numbers 2459322 to 2460668 of the Escherichia coli genomic sequence (GenBank Accession No. U00096).

The “fadD gene” can mean a gene coding for an enzyme having the fatty acyl-CoA synthetase activity, which generates a fatty acyl-CoA from a long chain fatty acid, and taking up it through the inner membrane, and which is found in the Enterobacteriaceae bacteria (Dirusso, C. C. and Black, P. N., 2004, J. Biol. Chem., 279:49563-49566; Schmelter, T. et al., 2004, J. Biol. Chem., 279: 24163-24170). Specific examples of gene encoding FadD include the gene located at the nucleotide numbers 1887770 to 1886085 (complementary strand) of the Escherichia coli genomic sequence (GenBank Accession No. U00096).

The “fadE gene” can mean a gene encoding an enzyme having the acyl-CoA dehydrogenase activity for oxidizing a fatty acyl-CoA, which is found in the Enterobacteriaceae bacteria (O'Brien, W. J. and Frerman, F. E. 1977, J. Bacteriol., 132:532-540; Campbell, J. W. and Cronan, J. E., 2002, J. Bacteriol., 184:3759-3764).

Specific examples of gene coding for FadE include the gene located at the nucleotide numbers 243303 to 240859 (complementary strand) of the Escherichia coli genomic sequence (GenBank Accession No. U00096).

The “fadB gene” can mean a gene coding for an enzyme constituting the α component of the fatty acid oxidation complex found in the Enterobacteriaceae bacteria and having four kinds of activities of enoyl-CoA hydratase, 3-hydroxyacyl-CoA dehydrogenase, 3-hydroxyacyl-CoA epimerase and Δ3-cis-Δ2-trans-enoyl-CoA isomerase (Pramanik, A. et al., 1979, J. Bacteriol., 137:469-473; Yang, S. Y. and Schulz, H., 1983, J. Biol. Chem., 258:9780-9785). Specific examples of gene coding for FadB include the gene located at the nucleotide numbers 4028994 to 4026805 (complementary strand) of the Escherichia coli genomic sequence (GenBank Accession No. U00096).

The “fadA gene” can mean a gene coding for an enzyme constituting the β component of the fatty acid oxidation complex found in the Enterobacteriaceae bacteria and having the 3-ketoacyl-CoA thiolase activity (Pramanik, A. et al., 1979, J. Bacteriol., 137: 469-473). Specific examples of gene coding for FadA include the gene located at the nucleotide numbers 4026795 to 4025632 (complementary strand) of the Escherichia coli genomic sequence (GenBank Accession No. U00096).

It is known that FadB and FadA form a complex in the fatty acid oxidation complex found in the Enterobacteriaceae bacteria, and the genes also form the fadBA operon (Yang, S. Y. et al., 1990, J. Biol. Chem., 265:10424-10429). Therefore, the entire operon can also be amplified as the fadBA operon.

The “fadJ gene” can mean a gene showing homology to the fadB gene and coding for an enzyme constituting the α component of the fatty acid oxidation complex functioning under an anaerobic condition and an aerobic condition (Campbell, J. W. et al., 2003, Mol. Microbiol., 47(3):793-805) and having four kinds of activities of enoyl-CoA hydratase, 3-hydroxyacyl-CoA dehydrogenase, 3-hydroxyacyl-CoA epimerase and Δ3-cis-Δ2-trans-enoyl-CoA isomerase (Pramanik, A. et al., 1979, J. Bacteriol., 137:469-473; Yang, S. Y. and Schulz, H., 1983, J. Biol. Chem., 258:9780-9785). Specific examples of gene coding for FadJ include the gene located at the nucleotide numbers 2457181 to 2455037 (complementary strand) of the Escherichia coli genomic sequence (GenBank Accession No. U00096).

The “fadI gene” can mean a gene showing homology to the fadA gene and coding for an enzyme constituting the β component of the fatty acid oxidation complex functioning under an anaerobic condition and an aerobic condition (Campbell, J. W. et al., 2003, Mol. Microbiol., 47(3):793-805) and having the 3-ketoacyl-CoA thiolase activity (Pramanik, A. et al., 1979, J. Bacteriol., 137: 469-473). Specific examples of gene coding for FadI include the gene located at the nucleotide numbers 2458491 to 2457181 (complementary strand) of the Escherichia coli genomic sequence (GenBank Accession No. U00096).

It is known that FadJ and FadI form a complex in the fatty acid oxidation complex found in the Enterobacteriaceae bacteria, and the genes also form the fadIJ operon (Yang, S. Y. et al., 1990, J. Biol. Chem., 265:10424-10429). Therefore, the entire operon can also be amplified as the fadIJ operon.

The ability to assimilate a hydrolysate of fats and oils or a fatty acid can also be enhanced by enhancing the cyo operon (cyoABCDE). The “cyoABCDE” can mean a group of genes coding for the subunits of the cytochrome bo terminal oxidase complex as one of the terminal oxidases found in the Enterobacteriaceae bacteria. The cyoB codes for the subunit I, cyoA codes for the subunit II, cyoC codes for the subunit III, cyoC codes for the subunit IV, and cyoE codes for an enzyme showing the heme O synthase activity (Gennis, R. B. and Stewart, V., 1996, pp. 217-261, In F. D. Neidhardt (ed.), Escherichia coli and Salmonella Cellular and Molecular Biology/Second Edition, American Society for Microbiology Press, Washington, D.C.; Chepuri et al., 1990, J. Biol. Chem., 265:11185-11192).

Specific examples of a gene coding for cyoA include the gene located at the nucleotide numbers 450834 to 449887 (complementary strand) of the Escherichia coli genomic sequence (GenBank Accession No. U00096) as the cyoA gene of Escherichia coli. Specific examples of a gene coding for cyoB include the gene located at the nucleotide numbers 449865 to 447874 (complementary strand) of the Escherichia coli genomic sequence (GenBank Accession No. U00096) as the cyoB gene of Escherichia coli. Specific examples of a gene coding for cyoC include the gene located at the nucleotide numbers 447884 to 447270 (complementary strand) of the Escherichia coli genomic sequence (GenBank Accession No. U00096) as the cyoC gene of Escherichia coli. Specific examples of a gene coding for cyoD include the gene located at the nucleotide numbers 447270 to 446941 (complementary strand) of the Escherichia coli genomic sequence (GenBank Accession No. U00096) as cyoD gene of Escherichia coli. Specific examples of a gene coding for cyoE include the gene located at the nucleotide numbers 446929 to 446039 (complementary strand) of the Escherichia coli genomic sequence (GenBank Accession No. U00096) as the cyoE gene of Escherichia coli.

The bacterium may be a strain which has been modified so that the activity of pyruvate synthase or pyruvate:NADP⁺ oxidoreductase is increased (refer to WO2009/031565).

The “pyruvate synthase” can mean an enzyme reversibly catalyzing the following reaction, which generates pyruvic acid from acetyl-CoA and CO₂ in the presence of an electron donor such as ferredoxin or flavodoxin (EC 1.2.7.1). Pyruvate synthase may be abbreviated as PS, and may also be designated pyruvate oxidoreductase, pyruvate ferredoxin oxidoreductase, pyruvate flavodoxin oxidoreductase, or pyruvate oxidoreductase. As the electron donor, ferredoxin or flavodoxin can be used.

Reduced ferredoxin+acetyl-CoA+CO₂->oxidized ferredoxin+pyruvic acid+CoA

Enhancement of the pyruvate synthase activity can be confirmed by preparing crude enzyme solutions from the microorganism before and after the enhancement, and comparing the before and after pyruvate synthase activities. The activity of pyruvate synthase can be measured by, for example, the method of Yoon et al. (Yoon, K. S. et al., 1997, Arch. Microbiol. 167:275-279). For example, the measurement can be attained by adding pyruvic acid to a reaction mixture containing oxidized methylviologen as an electron acceptor, CoA, and a crude enzyme solution, and spectroscopically measuring the amount of reduced methylviologen, which increases due to the decarboxylation of pyruvic acid. One unit (U) of the enzymatic activity is defined as an activity of reducing 1 μmol of methylviologen per 1 minute. When the parent strain has pyruvate synthase activity, the activity can be increased, for example, 1.5 times or more, 2 times or more, or 3 times or more, as compared to that of the parent strain. When the parent strain does not have the pyruvate synthase activity, although it is sufficient that pyruvate synthase is produced by the introduction of the pyruvate synthase gene, the activity can be enhanced to such an extent that the enzymatic activity can be measured, and the activity can be 0.001 U/mg (cell protein) or higher, 0.005 U/mg or higher, or 0.01 U/mg or higher. The pyruvate synthase is sensitive to oxygen, and activity expression and measurement thereof are generally often difficult (Buckel, W. and Golding, B. T., 2006, Ann. Rev. of Microbiol., 60:27-49). Therefore, when the enzymatic activity is measured, the enzymatic reaction can be performed with reducing oxygen concentration in a reaction vessel.

As the gene encoding pyruvate synthase, it is possible to use pyruvate synthase genes of bacteria having the reductive TCA cycle such as Chlorobium tepidum and Hydrogenobacter thermophilus. Moreover, it is also possible to use pyruvate synthase genes of bacteria belonging to the family Eenterobacteriaceae including Escherichia coli. Furthermore, as the gene coding for pyruvate synthase, pyruvate synthase genes of autotrophic methanogens such as Methanococcus maripaludis, Methanocaldococcus jannaschii, and Methanothermobacter thermautotrophicus can be used.

The “pyruvate:NADP⁺ oxidoreductase” can mean an enzyme reversibly catalyzing the following reaction, which generates pyruvic acid from acetyl CoA and CO₂, in the presence of an electron donor such as NADPH or NADH (EC 1.2.1.15). The pyruvate:NADP⁺ oxidoreductase may be abbreviated as PNO, and may also be designated pyruvate dehydrogenase. However, the “pyruvate dehydrogenase activity” can be the activity for catalyzing the oxidative decarboxylation of pyruvic acid to generate acetyl-CoA, as described later, and pyruvate dehydrogenase (PDH) which catalyses this reaction is an enzyme different from pyruvate:NADP⁺ oxidoreductase. The pyruvate:NADP⁺ oxidoreductase can use NADPH or NADH as the electron donor.

NADPH+acetyl-CoA+CO₂->NADP⁺+pyruvic acid+CoA

Enhancement of the pyruvate:NADP⁺ oxidoreductase activity can be confirmed by preparing crude enzyme solutions from the microorganism before the enhancement and the microorganism after the enhancement, and comparing the pyruvate:NADP⁺ oxidoreductase activities of them. The activity of pyruvate:NADP⁺ oxidoreductase can be measured by, for example, the method of Inui et al. (Inui, H., et al., 1987, J. Biol. Chem., 262:9130-9135). For example, the measurement can be attained by adding pyruvic acid to a reaction mixture containing oxidized methylviologen as an electron acceptor, CoA, and a crude enzyme solution, and spectroscopically measuring the amount of reduced methylviologen, which increases due to the decarboxylation of pyruvic acid. One unit (U) of the enzymatic activity is defined as an activity of reducing 1 μmol of methylviologen per 1 minute. When the parent strain has the pyruvate:NADP⁺ oxidoreductase activity, the activity can increase 1.5 times or more, 2 times or more, or 3 times or more, as compared to that of the parent strain. When the parent strain does not have the pyruvate:NADP⁺ oxidoreductase activity, although it is sufficient that pyruvate:NADP⁺ oxidoreductase is produced by the introduction of the pyruvate:NADP⁺oxidoreductase gene, the activity can be enhanced to such an extent that the enzymatic activity can be measured, and the activity can be 0.001 U/mg (cell protein) or higher, 0.005 U/mg or higher, or 0.01 U/mg or higher. The pyruvate:NADP⁺ oxidoreductase is sensitive to oxygen, and activity expression and measurement thereof are generally often difficult (Inui, H., et al, 1987, J. Biol. Chem., 262: 9130-9135; Rotte, C. et al., 2001, Mol. Biol. Evol., 18:710-720).

As for the gene coding for pyruvate:NADP⁺ oxidoreductase, it is known that, besides the pyruvate:NADP⁺ oxidoreductase gene of Euglena gracilis, which is a photosynthetic eukaryotic microorganism and is also classified into protozoans (Nakazawa, M. et al., 2000, FEBS Lett., 479:155-156), and the pyruvate:NADP⁺ oxidoreductase gene of a protist, Cryptosporidium parvum (Rotte, C. et al., 2001, Mol. Biol. Evol., 18:710-720), a homologous gene also exists in Bacillariophyta, Tharassiosira pseudonana (Ctrnacta, V. et al., 2006, J. Eukaryot. Microbiol., 53:225-231).

Specifically, the pyruvate:NADP⁺ oxidoreductase gene of Euglena gracilis can be used (GenBank Accession No. AB021127).

The microorganism may be a microorganism modified so that the pyruvate synthase activity is increased by a modification for increasing the activity of recycling the oxidized electron donor to reduced electron donor, which is required for the pyruvate synthase activity, as compared to a parent strain, for example, a wild-type or non-modified strain. Examples of the activity of recycling oxidized electron donor to reduced electron donor include the ferredoxin-NADP⁺ reductase activity. Furthermore, the microorganism may be a microorganism modified so that the activity of pyruvate synthase is increased by such a modification that pyruvate synthase activity increases, in addition to the enhancement of the electron donor recycling activity. The aforementioned parent strain may be a strain inherently having a gene coding for the electron donor recycling activity, or a strain which does not inherently have the electron donor recycling activity, but can be imparted with the activity by introduction of a gene coding for the activity to show improved L-amino acid-producing ability.

The “ferredoxin-NADP⁺ reductase” means an enzyme that reversibly catalyzes the following reaction (EC 1.18.1.2).

Reduced ferredoxin+NADP⁺->oxidized ferredoxin+NADPH+H⁺

This reaction is a reversible reaction, and can generate the reduced ferredoxin in the presence of NADPH and the oxidized ferredoxin. Ferredoxin is replaceable with flavodoxin, and the enzyme designated flavodoxin-NADP⁺ reductase also has an equivalent function. Existence of ferredoxin-NADP⁺ reductase is confirmed in a wide variety of organisms ranging from microorganisms to higher organisms (refer to Carrillo, N. and Ceccarelli, E. A., 2003, Eur. J. Biochem., 270:1900-1915; Ceccarelli, E. A. et al., 2004, Biochim. Biophys. Acta., 1698:155-165), and some of the enzymes are also named ferredoxin-NADP⁺ oxidoreductase or NADPH-ferredoxin oxidoreductase.

Enhancement of the ferredoxin-NADP⁺ reductase activity can be confirmed by preparing crude enzyme solutions from the microorganism before the modification and the microorganism after the modification, and comparing the ferredoxin-NADP⁺ reductase activities of them. The activity of ferredoxin-NADP⁺ reductase can be measured by, for example, the method of Blaschkowski et al. (Blaschkowski, H. P. et al., 1982, Eur. J. Biochem., 123:563-569). For example, the activity can be measured by using ferredoxin as a substrate to spectroscopically measure decrease of the amount of NADPH. One unit (U) of the enzymatic activity is defined as activity for oxidizing 1 μmol of NADPH per 1 minute. When the parent strain has the ferredoxin-NADP⁺ reductase activity, and the activity of the parent strain is sufficiently high, it is not necessary to enhance the activity. However, the enzymatic activity can be increased 1.5 times or more, 2 times or more, or 3 times or more, as compared to that of the parent strain.

Genes encoding the ferredoxin-NADP⁺ reductase are found in many biological species, and any of them showing the activity in the objective L-amino acid-producing strain can be used. As for Escherichia coli, the fpr gene has been identified as a gene of flavodoxin-NADP⁺ reductase (Bianchi, V. et al., 1993, 175:1590-1595). Moreover, it is known that, in Pseudomonas putida, an NADPH-putidaredoxin reductase gene and a putidaredoxin gene exist as an operon (Koga, H. et al., 1989, J. Biochem. (Tokyo), 106:831-836).

Examples of the flavodoxin-NADP⁺ reductase gene of Escherichia coli can include the fpr gene which is located at the nucleotide numbers 4111749 to 4112495 (complementary strand) of the genomic sequence of the Escherichia coli K-12 strain (GenBank Accession No. U00096). Moreover, a ferredoxin-NADP⁺ reductase gene (GenBank Accession No. BAB99777) is also found at the nucleotide numbers 2526234 to 2527211 of the genomic sequence of Corynebacterium glutamicum (GenBank Accession No. BA00036).

The pyruvate synthase activity requires presence of ferredoxin or flavodoxin as an electron donor. Therefore, the microorganism may be a microorganism modified so that the activity of pyruvate synthase is increased by such a modification that ferredoxin- or flavodoxin-producing ability is improved.

Moreover, the microorganism may also be modified so that the ferredoxin- or flavodoxin-producing ability is improved, in addition to being modified so that the pyruvate synthase activity or flavodoxin-NADP⁺ reductase and pyruvate synthase activities are enhanced.

“Ferredoxin” can refer to a protein bound with an iron-sulfur cluster containing nonheme iron atoms (Fe) and sulfur atoms, and called 4Fe-4S, 3Fe-4S or 2Fe-2S cluster, which functions as a one-electron carrier. “Flavodoxin” can refer to a protein containing FMN (flavin-mononucleotide) as a prosthetic group, which functions as a one- or two-electron carrier. Ferredoxin and flavodoxin are described in the reference of McLean et al. (McLean K. J. et al., 2005, Biochem. Soc. Trans., 33:796-801).

The parent strains to be subjected to the modification may be strains that inherently have an endogenous gene encoding ferredoxin or flavodoxin. Alternatively, the parent strains may be strains that do not inherently have a gene encoding ferredoxin or flavodoxin, but can be imparted with the activity by introduction of a ferredoxin or flavodoxin gene to show improved L-amino acid-producing ability.

Improvement of the ferredoxin- or flavodoxin-producing ability as compared to the parent strain such as a wild-type or non-modified strain can be confirmed by, for example, SDS-PAGE, two-dimensional electrophoresis or Western blotting using antibodies (Sambrook, J. et al., 1989, Molecular Cloning A Laboratory Manual/Second Edition, Cold Spring Harbor Laboratory Press, New York). Degree of the increase of the production amount is not particularly limited so long as it increases as compared to that of a wild-type strain or non-modified strain. However, it can increase, for example, 1.5 times or more, 2 times or more, or 3 times or more, as compared to that of a wild-type or non-modified strain.

The activities of ferredoxin and flavodoxin can be measured by adding them to an appropriate oxidation-reduction reaction system. For example, a method which includes reducing produced ferredoxin with ferredoxin-NADP⁺ reductase and quantifying reduction of cytochrome C by the produced reduced ferredoxin is disclosed by Boyer et al. (Boyer, M. E. et al., 2006, Biotechnol. Bioeng., 94:128-138). Furthermore, the activity of flavodoxin can be measured by the same method using flavodoxin-NADP⁺ reductase.

Genes encoding ferredoxin or flavodoxin are widely distributed, and any of those can be used so long as encoded ferredoxin or flavodoxin can be utilized by pyruvate synthase and an electron donor recycling system. For example, in Escherichia coli, the fdx gene exists as a gene encoding ferredoxin having a 2Fe-2S cluster (Ta, D. T. and Vickery, L. E., 1992, J. Biol. Chem., 267:11120-11125), and the yfhL gene is expected as a gene encoding ferredoxin having a 4Fe-4S cluster. Furthermore, as the flavodoxin gene, presence of the fldA gene (Osborne C. et al., 1991, J. Bacteriol., 173:1729-1737) and presence of the fldB gene (Gaudu, P. and Weiss, B., 2000, J. Bacteriol., 182:1788-1793) are known. In the genomic sequence of Corynebacterium glutamicum (GenBank Accession No. BA00036), multiple ferredoxin genes, fdx (GenBank Accession No. BAB97942) were found at the nucleotide numbers of 562643 to 562963, and the fer gene was found at the nucleotide numbers of 1148953 to 1149270 (GenBank Accession No. BAB98495). Furthermore, in the Chlorobium tepidum, many ferredoxin genes exist, and ferredoxin I and ferredoxin II have been identified as genes for the 4Fe-4S type ferredoxin, which serves as the electron acceptor of pyruvate synthase (Yoon, K. S. et al., 2001, J. Biol. Chem., 276:44027-44036). Ferredoxin or flavodoxin genes of bacteria having the reductive TCA cycle, such as Hydrogenobacter thermophilus, can also be used.

Specific examples of the ferredoxin gene of Escherichia coli include the fdx gene located at the nucleotide numbers of 2654770 to 2655105 (complementary strand) of the genomic sequence of the Escherichia coli K-12 strain (GenBank Accession No. U00096), and the yfhL gene located at the nucleotide numbers of 2697685 to 2697945 of the same.

The L-amino acid-producing bacterium may be modified for a gene involved in the glycerol metabolism.

As for genes involved in the glycerol metabolism, in order to enhance glycerol-assimilating ability, expression of the glpR gene (EP 1715056) may be attenuated, or expression of the glycerol metabolism genes (EP 1715055 A) such as glpA, glpB, glpC, glpD, glpE, glpF, glpG, glpK, glpQ, glpT, glpX, tpiA, gldA, dhaK, dhaL, dhaM, dhaR, fsa, and talC genes may be enhanced.

In particular, in order to enhance glycerol-assimilating ability, the glycerol dehydrogenase gene (gldA), and the PEP-dependent dihydroxyacetone kinase gene (dhaKLM) or the ATP-dependent dihydroxyacetone kinase gene (dak) can be enhanced in combination. Further, expression of the fructose-6-phosphate aldolase gene (fsaB) may be enhanced (WO2008/102861).

Furthermore, as for glycerol kinase (glpK), a glpK gene which has been modified to be desensitized to the feedback inhibition by fructose-1,6-phosphate (WO2008/081959, WO2008/107277) can be used.

The family Enterobacteriaceae encompasses bacteria belonging to the genera of Escherichia, Enterobacter, Erwinia, Klebsiella, Pantoea, Photorhabdus, Providencia, Salmonella, Serratia, Shigella, Morganella, Yersinia, and so forth. In particular, bacteria classified into the family Enterobacteriaceae according to the taxonomy used in the NCBI (National Center for Biotechnology Information) database (http://www.ncbi.nlm.nih.gov/Taxonomy/Browser/wwwtax.cgi?id=91347) are examples.

The bacterium belonging to the genus Escherichia that can be used is not particularly limited. However, examples include, for example, the bacteria of the phyletic groups described in the work of Neidhardt et al. (Neidhardt F. C. Ed., 1996, Escherichia coli and Salmonella: Cellular and Molecular Biology/Second Edition, pp. 2477-2483, Table 1, American Society for Microbiology Press, Washington, D.C.). Specific examples include the Escherichia coli W3110 (ATCC 27325), Escherichia coli MG1655 (ATCC 47076) and so forth derived from the prototype wild-type strain, K12 strain.

These strains are available from, for example, American Type Culture Collection (Address: P.O. Box 1549 Manassas, Va. 20108, United States of America). That is, accession numbers are given to each of the strains, and the strains can be ordered by using these numbers. The accession numbers of the strains are listed in the catalogue of the American Type Culture Collection. The same shall apply to the strains mentioned below with ATCC numbers.

A bacterium belonging to the genus Pantoea can mean that the bacterium is classified into the genus Pantoea according to the classification known to a person skilled in the art of microbiology. Some species of Enterobacter agglomerans have been recently re-classified into Pantoea agglomerans, Pantoea ananatis, Pantoea stewartii, or the like, based on the nucleotide sequence analysis of 16S rRNA, etc. (Int. J. Syst. Bacteriol., 1993, 43, 162-173). Bacteria belonging to the genus Pantoea can encompass such bacteria re-classified into the genus Pantoea as described above.

Typical strains of the Pantoea bacteria include Pantoea ananatis, Pantoea stewartii, Pantoea agglomerans, and Pantoea citrea. Specific examples include the following strains:

Pantoea ananatis AJ13355 strain (FERM BP-6614, European Patent Laid-open (EP-A) No. 0952221)

Pantoea ananatis AJ13356 strain (FERM BP-6615, EP 0952221 A)

Although these strains are described as Enterobacter agglomerans in EP 0952221 A, they are currently classified as Pantoea ananatis on the basis of nucleotide sequence analysis of the 16S rRNA etc., as described above.

Examples of the Enterobacter bacteria include Enterobacter agglomerans, Enterobacter aerogenes, and the like. Specifically, the strains exemplified in EP 952221 A can be used. Typical strains of the genus Enterobacter include the Enterobacter agglomerans ATCC 12287 strain.

Examples of the Erwinia bacteria include Erwinia amylovora and Erwinia carotovora, and examples of the Klebsiella bacteria include Klebsiella planticola. Specific examples include the following strains:

Erwinia amylovora ATCC 15580 strain

Erwinia carotovora ATCC 15713 strain

Klebsiella planticola AJ13399 strain (FERM BP-6600, EP 955368 A)

Klebsiella planticola AJ13410 strain (FERM BP-6617, EP 955368 A)

In the present invention, the “coryneform bacteria” can also include bacteria which have previously been classified into the genus Brevibacterium but are presently united into the genus Corynebacterium (Liebl and W. et al, 1991, Int. J. Syst. Bacteriol., 41:255-260), and bacteria belonging to the genus Brevibacterium, which are closely related to the genus Corynebacterium. Specific examples of such coryneform bacteria include the followings:

Corynebacterium acetoacidophilum

Corynebacterium acetoglutamicum

Corynebacterium alkanolyticum

Corynebacterium callunae

Corynebacterium glutamicum

Corynebacterium lilium

Corynebacterium melassecola

Corynebacterium thermoaminogenes (Corynebacterium efficiens)

Corynebacterium herculis

Brevibacterium divaricatum

Brevibacterium flavum

Brevibacterium immariophilum

Brevibacterium lactofermentum (Corynebacterium glutamicum)

Brevibacterium roseum

Brevibacterium saccharolyticum

Brevibacterium thiogenitalis

Corynebacterium ammoniagenes

Brevibacterium album

Brevibacterium cerinum

Microbacterium ammoniaphilum

Specific examples of these bacteria include the following strains:

Corynebacterium acetoacidophilum ATCC 13870

Corynebacterium acetoglutamicum ATCC 15806

Corynebacterium alkanolyticum ATCC 21511

Corynebacterium callunae ATCC 15991

Corynebacterium glutamicum ATCC 13020, ATCC 13032, ATCC 13060

Corynebacterium lilium ATCC 15990

Corynebacterium melassecola ATCC 17965

Corynebacterium thermoaminogenes AJ12340 (FERM BP-1539)

Corynebacterium herculis ATCC 13868

Brevibacterium divaricatum ATCC 14020

Brevibacterium flavum ATCC 13826, ATCC 14067

Brevibacterium immariophilum ATCC 14068

Brevibacterium lactofermentum ATCC 13869 (Corynebacterium glutamicum ATCC 13869)

Brevibacterium roseum ATCC 13825

Brevibacterium saccharolyticum ATCC 14066

Brevibacterium thiogenitalis ATCC 19240

Brevibacterium ammoniagenes ATCC 6871, ATCC 6872

Brevibacterium album ATCC 15111

Brevibacterium cerinum ATCC 15112

Microbacterium ammoniaphilum ATCC 15354

The bacterium having an amino acid-producing ability can refer to a bacterium having an ability to produce an L-amino acid and secrete it in a medium when it is cultured in the medium, preferably such that an amount of 0.5 g/L or more, or 1.0 g/L or more of the L-amino acid is produced. The L-amino acid can include L-alanine, L-arginine, L-asparagine, L-asparatic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, and L-valine. L-Threonine, L-lysine, and L-glutamic acid are particularl examples.

Methods for imparting an L-amino acid-producing ability to such bacteria as mentioned above and methods for enhancing an L-amino acid-producing ability of such bacteria as mentioned above are described below.

To impart the ability to produce an L-amino acid, methods conventionally employed in the breeding of coryneform bacteria or bacteria of the genus Escherichia (see “Amino Acid Fermentation”, Gakkai Shuppan Center (Ltd.), 1st Edition, published May 30, 1986, pp. 77-100) can be used. Such methods can include acquiring an auxotrophic mutant, an L-amino acid analogue-resistant strain, or a metabolic regulation mutant, or constructing a recombinant strain in which an L-amino acid biosynthesis enzyme is overexpressed. In the breeding of L-amino acid-producing bacteria, one or more of the above-described properties such as auxotrophy, analogue resistance, and metabolic regulation mutation can be imparted. The expressions of one or two or more L-amino acid biosynthesis enzymes can be enhanced. Furthermore, the methods of imparting properties such as an auxotrophy, analogue resistance, or metabolic regulation mutation can be combined with enhancement of the biosynthesis enzymes.

An auxotrophic mutant strain, L-amino acid analogue-resistant strain, or metabolic regulation mutant strain with the ability to produce an L-amino acid can be obtained by subjecting a parent or wild-type strain to conventional mutatagenesis, such as exposure to X-rays or UV irradiation, or treatment with a mutagen such as N-methyl-N′-nitro-N-nitrosoguanidine, and then selecting those which exhibit autotrophy, analogue resistance, or a metabolic regulation mutation and which also have the ability to produce an L-amino acid.

Moreover, the L-amino acid-producing ability can also be imparted or enhanced by increasing the enzymatic activity by gene recombination. An example of the method for increasing enzymatic activity can include modifying the bacterium so that the expression of a gene coding for an enzyme involved in the biosynthesis of an L-amino acid is enhanced. Gene expression can also be increased by introducing an amplification plasmid prepared by introducing a DNA fragment containing the gene into an appropriate plasmid such as a plasmid vector which contains, for example, at least a gene responsible for replication and proliferation functions of the plasmid in the microorganism, increasing the copy number of the gene on the chromosome by conjugation, transfer, or the like, or introducing a mutation into the promoter region of the gene (refer to International Publication WO95/34672).

When an objective gene is introduced into the aforementioned amplification plasmid or chromosome, any promoter can be used to express the gene so long as the chosen promoter functions in bacteria belonging to the coryneform bacteria. The promoter can be the native promoter for the gene, or a modified promoter. The expression of a gene can also be controlled by suitably choosing a promoter that strongly functions in bacteria belonging to coryneform bacteria, or by making the −35 and −10 regions of the promoter closer to the consensus sequence. These methods for enhancing expression of enzyme genes are fully described in International Publication WO00/18935, EP 1010755 A, and so forth.

Specific methods for imparting an L-amino acid-producing ability to bacteria and bacteria imparted with L-amino acid-producing ability are exemplified below.

L-Threonine-Producing Bacteria

Examples of microorganisms having L-threonine-producing ability can include bacteria in which one or more activities of L-threonine biosynthesis system enzymes are enhanced. Examples of L-threonine biosynthetic enzymes include aspartokinase III (lysC), aspartate semialdehyde dehydrogenase (asd), aspartokinase I (thrA), homoserine kinase (thrB), threonine synthase (thrC) encoded by the thr operon, and aspartate aminotransferase (aspartate transaminase) (aspC). The names of the genes coding for the respective enzymes are mentioned in the parentheses after the names of the enzymes (the same shall apply throughout this specification). Among these enzymes, aspartate semialdehyde dehydrogenase, aspartokinase I, homoserine kinase, aspartate aminotransferase, and threonine synthase are particular examples. The genes coding for the L-threonine biosynthetic enzymes can be introduced into an Escherichia bacterium which has a reduced ability to decompose threonine. An example of such an Escherichia bacterium having a reduced ability to decompose threonine is the TDH6 strain which is deficient in threonine dehydrogenase activity (Japanese Patent Laid-open No. 2001-346578).

The enzymatic activities of the L-threonine biosynthetic enzymes are inhibited by the endproduct, L-threonine. Therefore, for constructing L-threonine-producing strains, it is desirable that the genes for the L-threonine biosynthetic enzymes are modified so that the enzymes are desensitized to feedback inhibition by L-threonine in the L-threonine-producing strains. The aforementioned thrA, thrB, and thrC genes constitute the threonine operon, which forms an attenuator structure. The expression of the threonine operon is inhibited by isoleucine and threonine in the culture medium and also suppressed by attenuation. Therefore, the threonine operon can be modified by removing the leader sequence in the attenuation region or the attenuator (refer to Lynn, S. P., Burton, W. S., Donohue, T. J., Gould, R. M., Gumport, R. L, and Gardner, J. F., J. Mol. Biol. 194:59-69 (1987); WO02/26993; WO2005/049808).

The native promoter of the threonine operon is present upstream of the threonine operon, and can be replaced with a non-native promoter (refer to WO98/04715), or a threonine operon which has been modified so that expression of the threonine biosynthesis gene is controlled by the repressor and promoter of λ-phage can be constructed (EP 0593792). Furthermore, in order to modify a bacterium so that it is desensitized to feedback inhibition by L-threonine, a strain resistant to α-amino-β-hydroxyisovaleric acid (AHV) can be selected.

It is prefereable that the copy number of the threonine operon that is modified for desensitization to the feedback inhibition by L-threonine is increased in the host, or the expression of the threonine operon is increased by ligating it to a potent promoter. The copy number can also be increased by, besides amplification using a plasmid, transferring the threonine operon to a genome using a transposon, Mu-phage, or the like.

Other than increasing expression of the L-threonine biosynthetic genes, expression of the genes involved in the glycolytic pathway, TCA cycle, or respiratory chain, the genes that regulate the expression of these genes, or the genes involved in sugar uptake can also be preferably increased. Examples of such genes effective for L-threonine production include the genes encoding transhydrogenase (pntAB, EP 733712 B), phosphoenolpyruvate carboxylase (pepC, WO95/06114), phosphoenolpyruvate synthase (pps, EP 877090 B), and a gene encoding pyruvate carboxylase from coryneform bacterium or Bacillus bacterium (WO99/18228, EP 1092776 A).

Resistance to L-threonine, L-homoserine, or both can be imparted to the host by, for example, enhancing expression of a gene that imparts resistance to L-threonine or L-homoserine. Examples of these genes include rhtA gene (Livshits, V. A. et al., 2003, Res. Microbiol., 154:123-135), rhtB (EP 0994190 A), rhtC gene (EP 1013765 A), yfiK, and yeaS genes (EP 1016710 A). The methods for imparting L-threonine resistance to a host are described in EP 0994190 A and WO90/04636.

Examples of L-threonine-producing bacteria and parent strains which can be used to derive such bacteria include, but are not limited to, strains belonging to the genus Escherichia, such as E. coli TDH-6/pVIC40 (VKPM B-3996) (U.S. Pat. No. 5,175,107, U.S. Pat. No. 5,705,371), E. coli 472T23/pYN7 (ATCC 98081) (U.S. Pat. No. 5,631,157), E. coli NRRL-21593 (U.S. Pat. No. 5,939,307), E. coli FERM BP-3756 (U.S. Pat. No. 5,474,918), E. coli FERM BP-3519 and FERM BP-3520 (U.S. Pat. No. 5,376,538), E. coli MG442 (Gusyatiner et al., Genetika (in Russian), 14, 947-956 (1978)), E. coli VL643 and VL2055 (EP 1149911 A) and so forth.

The TDH-6 strain is deficient in the thrC gene, as well as being sucrose-assimilative, and the ilvA gene has a leaky mutation. This strain also has a mutation in the rhtA gene, which imparts resistance to high concentration of threonine or homoserine. The B-3996 strain contains the plasmid pVIC40, which was obtained by inserting the thrA*BC operon, including a mutant thrA gene, into the RSF1010-derived vector. This mutant thrA gene encodes aspartokinase homoserine dehydrogenase I which is substantially desensitized to feedback inhibition by threonine. The B-3996 strain was deposited on Nov. 19, 1987 at the All-Union Scientific Center of Antibiotics (Nagatinskaya Street 3-A, 117105 Moscow, Russia) under the accession number RIA 1867. The strain was also deposited at the Russian National Collection of Industrial Microorganisms (VKPM) (1 Dorozhny proezd., 1 Moscow 117545, Russia) on Apr. 7, 1987 under the accession number VKPM B-3996.

E. coli VKPM B-5318 (EP 0593792 B) can also be used as an L-threonine-producing bacterium or a parent strain for deriving it. The B-5318 strain is prototrophic with regard to isoleucine, and a temperature-sensitive lambda-phage Cl repressor and PR promoter replace the regulatory region of the threonine operon in the plasmid pVIC40. The VKPM B-5318 strain was deposited as an international deposit at the Russian National Collection of Industrial Microorganisms (VKPM) (1 Dorozhny proezd., 1 Moscow 117545, Russia) on May 3, 1990 under the accession number of VKPM B-5318.

The thrA gene which encodes aspartokinase homoserine dehydrogenase I of Escherichia coli is located at the nucleotide numbers 337 to 2,799 on the genome sequence of the Escherichia coli MG1655 strain registered under GenBank Accession No. U00096, and coding for the protein registered under GenBank accession No. AAC73113. The thrB gene which encodes homoserine kinase of Escherichia coli is located at the nucleotide numbers 2,801 to 3,733 on the genome sequence of the Escherichia coli MG1655 strain registered under GenBank Accession No. U00096, and coding for the protein registered under GenBank accession No. AAC73114. The thrC gene which encodes threonine synthase of Escherichia coli is located at the nucleotide numbers 3,734 to 5,020 on the genome sequence of the Escherichia coli MG1655 strain registered under GenBank Accession No. U00096, and coding for the protein registered under GenBank accession No. AAC73115. These three genes are encoded as the threonine operon consisting of thrLABC downstream of the thrL gene coding for the leader peptide. To enhance expression of the threonine operon, it is effective to remove the attenuator region which affects the transcription from the operon (WO2005/049808, WO2003/097839).

A mutant thrA gene which codes for aspartokinase homoserine dehydrogenase I resistant to feedback inhibition by threonine, as well as the thrB and thrC genes can be obtained as one operon from the well-known pVIC40 plasmid, which is present in the threonine-producing E. coli strain VKPM B-3996. pVIC40 is described in detail in U.S. Pat. No. 5,705,371.

The rhtA gene was obtained as being able to impart resistance to homoserine and threonine (rht: resistant to threonine/homoserine), is located at the nucleotide numbers 848,433 to 849,320 (complementary strand) on the genome sequence of the Escherichia coli MG1655 strain registered under GenBank Accession No. U00096, and codes for the protein registered under GenBank accession No. AAC73900. Also, it was revealed that the rhtA23 mutation, which improves expression of rhtA, is an A-for-G substitution at position −1 with respect to the ATG start codon (Livshits, V. A. et al., 2003, Res. Microbiol., 154:123-135, EP 1013765 A).

The asd gene of E. coli is located at the nucleotide numbers 3,571,798 to 3,572,901 (complementary strand) on the genome sequence of the Escherichia coli MG1655 strain registered under GenBank Accession No. U00096, and codes for the protein registered under GenBank accession No. AAC76458. It can be obtained by PCR (refer to White, T. J. et al., Trends Genet, 5, 185 (1989)) utilizing primers prepared based on the nucleotide sequence of the gene. The asd genes of other microorganisms can also be obtained in a similar manner.

The aspC gene of E. coli is located at the nucleotide numbers 983,742 to 984,932 (complementary strand) on the genome sequence of the Escherichia coli MG1655 strain registered under GenBank Accession No. U00096, and codes for the protein registered under GenBank accession No. AAC74014, and can be obtained by PCR. The aspC genes of other microorganisms can also be obtained in a similar manner.

L-Lysine-Producing Bacteria

L-Lysine-producing bacteria and methods for constructing them are exemplified below.

Examples of strains having L-lysine-producing ability include, for example, L-lysine analogue-resistant strains and metabolic regulation mutant strains. Examples of L-lysine analogue include, but are not limited to, oxalysine, lysine hydroxamate, S-(2-aminoethyl)-L-cysteine (also abbreviated as “AEC” hereinafter), γ-methyllysine, α-chlorocaprolactam and so forth. Mutant strains having resistance to these lysine analogues can be obtained by subjecting a bacterium belonging to the family Enterobacteriaceae or a coryneform bacterium to a conventional artificial mutagenesis treatment. Specific examples of L-lysine-producing bacteria include Escherichia coli AJ11442 (FERM BP-1543, NRRL B-12185, see Japanese Patent Laid-open No. 56-18596 and U.S. Pat. No. 4,346,170), Escherichia coli VL611 strain (Japanese Patent Laid-open No. 2000-189180), and so forth. As an L-lysine-producing Escherichia coli, the WC196 strain may also be used (see International Publication WO96/17930).

Further, an L-lysine-producing bacterium can also be constructed by increasing activity of an L-lysine biosynthesis system enzyme. Increase of activity of such an enzyme can be attained by increasing the copy number of the gene coding for the enzyme in cells, or by modifying an expression control sequence thereof.

A gene can be modified to enhance expression by, for example, increasing the copy number of the gene in the cells by means of genetic recombination techniques. For example, a recombinant DNA can be prepared by ligating a DNA fragment containing the gapA gene with a vector, such as a multi-copy vector, which is able to function in a host microorganism, and introduced into a bacterium to transform it.

Increase of copy number of a gene can also be achieved by introducing multiple copies of the gene into a genomic DNA of a bacterium. In order to introduce multiple copies of a gene into a genomic DNA of a bacterium, homologous recombination is carried out by using a sequence which is present in multiple copies in the genomic DNA as targets. Examples of such sequences in genomic DNA include repetitive DNA, and inverted repeats existing at the end of a transposable element. Another gene may be introduced aside the gapA gene existing on a genome in tandem, or it may be introduced into an unnecessary gene on a genome in a plural number. Such gene transfer can be attained by using a temperature sensitive vector or an integration vector.

Alternatively, as disclosed in Japanese Patent Laid-open No. 2-109985, it is also possible to incorporate the gene into a transposon, and allow it to transfer to introduce multiple copies of the genes into a genomic DNA. Transfer of the gene to the genome can be confirmed by performing Southern hybridization using a part of the gene as a probe.

Further, in addition to the aforementioned increase of the gene copy number, expression of gene can be enhanced by replacing an expression control sequence such as a promoter of the gene on a genome DNA or plasmid with a stronger one, by making the −35 and −10 regions of the gene closer to the consensus sequence, by amplifying a regulator that increases expression of the gene, or by deleting or attenuating a regulator that decreases expression of the gene according to the methods described in International Publication WO00/18935. For example, the lac promoter, trp promoter, trc promoter, tac promoter, araBA promoter, lambda phage PR promoter and PL promoter, tet promoter, T7 promoter, Φ10 promoter, and so forth are known as strong promoters. A promoter or SD region of the gapA gene can also be modified so as to become stronger by introducing a nucleotide substitution or the like. Examples of methods for evaluating strength of a promoter and strong promoters are described in the paper of Goldstein et al. (Prokaryotic promoters in biotechnology, Biotechnol. Annu Rev., 1995, 1, 105-128) and so forth. In addition, it is known that substitution of several nucleotides in a spacer region between the ribosome binding site (RBS) and translation initiation codon, especially a sequence immediately upstream from the initiation codon, greatly affects mRNA translation efficiency, and therefore this sequence may be modified. Expression control regions such as promoter of a gene may also be identified by using a promoter search vector or gene analysis software such as GENETYX. By such substitution or modification of promoter as described above, expression of a gene is enhanced. Substitution of an expression control sequence can also be attained by, for example, a method using a temperature sensitive plasmid or Red-driven integration (WO2005/010175).

Examples of genes coding for L-lysine biosynthetic enzymes include genes coding for enzymes of the diaminopimelate pathway such as dihydrodipicolinate synthase gene (dapA), aspartokinase gene (lysC), dihydrodipicolinate reductase gene (dapB), diaminopimelate decarboxylase gene (lysA), diaminopimelate dehydrogenase gene (ddh) (WO96/40934 for all the foregoing genes), phosphoenolpyrvate carboxylase gene (ppc) (Japanese Patent Laid-open No. 60-87788), aspartate aminotransferase gene (aspC) (Japanese Patent Publication (Kokoku) No. 6-102028), diaminopimelate epimerase gene (dapF) (Japanese Patent Laid-open No. 2003-135066), and aspartate semialdehyde dehydrogenease gene (asd) (WO00/61723), and genes coding for enzymes of the aminoadipic acid pathway such as homoaconitate hydratase gene (Japanese Patent Laid-open No. 2000-157276). In addition, the parent strain may show an increased level of expression of the gene involved in energy efficiency (cyo) (EP 1170376 A), the gene coding for nicotinamide nucleotide transhydrogenase (pntAB) (U.S. Pat. No. 5,830,716), the ybjE gene coding for a protein having L-lysine excretion activity (WO2005/073390), the gene coding for glutamate dehydrogenase (gdhA) (Valle F. et al., 1983, Gene 23:199-209), or an arbitrary combination of these. Abbreviations for the genes are shown in the parentheses.

It is known that the wild-type dihydrodipicolinate synthase derived from Escherichia coli suffers from feedback inhibition by L-lysine, and it is known that the wild-type aspartokinase derived from Escherichia coli suffers from suppression and feedback inhibition by L-lysine. Therefore, when the dapA gene and lysC gene are used, these genes are preferably genes coding for mutant enzymes desensitized to the feedback inhibition by L-lysine.

Examples of DNA encoding a mutant dihydrodipicolinate synthetase desensitized to feedback inhibition by L-lysine include a DNA encoding such a protein having an amino acid sequence in which the histidine residue at the position 118 is replaced by tyrosine residue. Examples of DNA encoding a mutant aspartokinase desensitized to feedback inhibition by L-lysine include a DNA encoding an AKIII having an amino acid sequence in which the threonine residue at the position 352, the glycine residue at the position 323, and the methionine residue at the position 318 are replaced by isoleucine, asparagine and isoleucine residues, respectively (for these mutants, see U.S. Pat. Nos. 5,661,012 and 6,040,160). Such mutant DNAs can be obtained by site-specific mutagenesis using PCR or the like.

Wide host-range plasmids RSFD80, pCAB1, and pCABD2 are known as plasmids containing a mutant dapA gene encoding a mutant dihydrodipicolinate synthase and a mutant lysC gene encoding a mutant aspartokinase (U.S. Pat. No. 6,040,160). Escherichia coli JM109 strain transformed with the plasmid was named AJ12396 (U.S. Pat. No. 6,040,160), and the strain was deposited at the National Institute of Bioscience and Human-Technology, Agency of Industrial Science and Technology, Ministry of International Trade and Industry (currently National Institute of Advanced Industrial Science and Technology, International Organism Depositary) on Oct. 28, 1993 and assigned an accession number of FERM P-13936, and the deposit was then converted to an international deposit under the provisions of Budapest Treaty on Nov. 1, 1994 and assigned an accession number of FERM BP-4859. RSFD80 can be obtained from the AJ12396 strain by a conventional method.

Examples of such enzymes involved in the L-lysine production include homoserine dehydrogenase, lysine decarboxylase (cadA, ldcC), malic enzyme, and so forth, and strains in which activities of these enzymes are decreased or deleted are disclosed in WO95/23864, WO96/17930, WO2005/010175, and so forth.

It is preferred that expressions of both the cadA and ldcC genes encoding lysine decarboxylase are decreased in order to decrease or delete the lysine decarboxylase activity. Expression of the both genes can be decreased by, for example, the method described in WO2006/078039.

In order to reduce or eliminate activities of these enzymes, a mutation may be introduced into genes of the enzymes on a genome by a usual mutagenesis method or gene recombination technique so that intracellular activities of the enzymes are reduced or eliminated. Such introduction of a mutation can be achieved by, for example, using genetic recombination to eliminate the genes coding for the enzymes on the genome or to modify an expression control sequence such as a promoter or the Shine-Dalgarno (SD) sequence. It can also be achieved by introducing a mutation for amino acid substitution (missense mutation), a stop codon (nonsense mutation), or a frame shift mutation for adding or deleting one or two nucleotides into regions coding for the enzymes on the genome, or partially or totally deleting the genes (Wang, J. P. et al., 2006, J. Agric. Food Chem., 54:9405-9410; Winkler W. C., 2005, Curr. Opin. Chem. Biol., 9:594-602; Qiu Z. and Goodman M. F., 1997, J. Biol. Chem., 272:8611-8617; Wente, S. R. and Schachman, H. K., 1991, J. Biol. Chem., 266:20833-20839). The enzymatic activities can also be decreased or eliminated by constructing a gene coding for a mutant enzyme, of which coding region is totally or partially deleted, and substituting it for a normal gene on a genome by homologous recombination or the like, or by introducing a transposon or IS factor into the gene.

For example, in order to introduce a mutation that decreases or eliminates the activities of the above-mentioned enzymes by genetic recombination, the following methods are used. A mutant gene is prepared by modifying a partial sequence of an objective gene so that it does not encode an enzyme that can function normally, and then a bacterium belonging to the family Enterobacteriaceae can be transformed with a DNA containing the mutant gene to cause recombination of a corresponding gene on the genome with the mutant gene to substitute the mutant gene for the objective gene on the genome. Examples of such gene substitution using homologous recombination include methods of using a linear DNA such as the method called Red-driven integration (Datsenko, K. A, and Wanner, B. L., 2000, Proc. Natl. Acad. Sci. USA, 97:6640-6645), and the method utilizing the Red driven integration in combination with an excisive system derived from λ phage (Cho, E. H., Gumport, R. I., Gardner, J. F., 2002, J. Bacteriol., 184:5200-5203) (refer to WO2005/010175), a method of using a plasmid containing a temperature sensitive replication origin (U.S. Pat. No. 6,303,383, Japanese Patent Laid-open No. 05-007491), and so forth. Further, such site-specific mutagenesis based on gene substitution using homologous recombination can also be performed by using a plasmid which is not able to replicate in a host.

Examples of L-lysine-producing bacteria include Escherichia coli WC196AcadAΔldc/pCABD2 (WO2006/078039). The strain was constructed by introducing the plasmid pCABD2 containing lysine biosynthesis genes (U.S. Pat. No. 6,040,160) into the WC 196 strain having disrupted cadA and ldcC genes, which encode lysine decarboxylase. The WC196 strain was bred from the W3110 strain, which was derived from Escherichia coli K-12, by replacing the wild type lysC gene on the chromosome of the W3110 strain with a mutant lysC gene encoding a mutant aspartokinase III in which threonine at position 352 was replaced with isoleucine, resulting in desensitization of the feedback inhibition thereof by L-lysine (U.S. Pat. No. 5,661,012), and conferring AEC resistance to the resulting strain (U.S. Pat. No. 5,827,698). The WC196 strain was designated Escherichia coli AJ13069, deposited at the National Institute of Bioscience and Human-Technology, Agency of Industrial Science and Technology (currently National Institute of Advanced Industrial Science and Technology, International Patent Organism Depositary, Tsukuba Central 6, 1-1, Higashi 1-Chome, Tsukuba-shi, Ibaraki-ken, 305-8566, Japan) on Dec. 6, 1994, and assigned an accession number of FERM P-14690. Then, it was converted to an international deposit under the provisions of the Budapest Treaty on Sep. 29, 1995, and assigned an accession number of FERM BP-5252 (U.S. Pat. No. 5,827,698). The WC196ΔcadAΔldc strain itself is also a preferred L-lysine-producing bacterium. The WC196ΔcadAΔldc was designated AJ110692, and deposited at the independent administrative agency, National Institute of Advanced Industrial Science and Technology, International Patent Organism Depositary (Tsukuba Central 6, 1-1, Higashi 1-Chome, Tsukuba-shi, Ibaraki-ken, 305-8566, Japan) on Oct. 7, 2008 as an international deposit and assigned an accession number of FERM BP-11027.

The plasmid pCABD2 contains a mutant dapA gene derived from Escherichia coli and coding for a dihydrodipicolinate synthase (DDPS) having a mutation for desensitization to the feedback inhibition by L-lysine, a mutant lysC gene derived from Escherichia coli and coding for aspartokinase III having a mutation for desensitization to the feedback inhibition by L-lysine, the dapB gene derived from Escherichia coli and coding for dihydrodipicolinate reductase, and the ddh gene derived from Brevibacterium lactofermentum and coding for diaminopimelate dehydrogenase (International Publications WO95/16042 and WO01/53459).

The procedures described above for enhancing gene expression of the enzymes involved in the L-lysine biosynthesis, and the methods for reducing the enzymatic activities can similarly be applied to genes coding for other L-amino acid biosynthesis enzymes.

Examples of L-lysine producing coryneform bacteria include AEC-resistant mutant strains (Brevibacterium lactofermentum AJ11082 (NRRL B-11470) strain etc., refer to Japanese Patent Publication Nos. 56-1914, 56-1915, 57-14157, 57-14158, 57-30474, 58-10075, 59-4993, 61-35840, 62-24074, 62-36673, 5-11958, 7-112437 and 7-112438); mutant strains requiring an amino acid such as L-homoserine for their growth (refer to Japanese Patent Publication Nos. 48-28078 and 56-6499); mutant strains showing resistance to AEC and further requiring an amino acid such as L-leucine, L-homoserine, L-proline, L-serine, L-arginine, L-alanine and L-valine (refer to U.S. Pat. Nos. 3,708,395 and 3,825,472); L-lysine-producing mutant strains showing resistance to DL-α-amino-ε-caprolactam, α-amino-lauryllactam, aspartic acid analogue, sulfa drug, quinoid and N-lauroylleucine; L-lysine-producing mutant strains showing resistance to oxaloacetate decarboxylase or a respiratory tract enzyme inhibitor (Japanese Patent Laid-open Nos. 50-53588, 50-31093, 52-102498, 53-9394, 53-86089, 55-9783, 55-9759, 56-32995, 56-39778, Japanese Patent Publication Nos. 53-43591 and 53-1833); L-lysine-producing mutant strains requiring inositol or acetic acid (Japanese Patent Laid-open Nos. 55-9784 and 56-8692); L-lysine-producing mutant strains that are susceptible to fluoropyruvic acid or a temperature of 34° C. or higher (Japanese Patent Laid-open Nos. 55-9783 and 53-86090); L-lysine-producing mutant strains of Brevibacterium or Corynebacterium bacteria showing resistance to ethylene glycol (U.S. Pat. No. 4,411,997) and so forth.

L-Cysteine-Producing Bacteria

Examples of L-cysteine-producing bacteria and parent strains for deriving them include, but not limited to, Escherichia bacteria such as E. coli JM15 transformed with multiple kinds of cysE gene alleles encoding serine acetyltransferase resistant to feedback inhibition (U.S. Pat. No. 6,218,168, Russian Patent Application No. 2003121601), E. coli W3110 in which a gene encoding a protein suitable for excretion of cytotoxic substances is overexpressed (U.S. Pat. No. 5,972,663), E. coli strain having decreased cysteine desulfhydrase activity (Japanese Patent Laid-open No. 11-155571), and E. coli W3110 in which activity of the positive transcriptional control factor of the cysteine regulon encoded by the cysB gene is increased (WO01/27307).

L-Leucine-Producing Bacteria

Examples of L-leucine-producing bacteria and parent strains which can be used to derive L-leucine-producing bacteria include, but are not limited to, Escherichia bacterial strains, such as E. coli strains resistant to leucine (for example, the 57 strain (VKPM B-7386, U.S. Pat. No. 6,124,121)) or leucine analogues including β-2-thienylalanine, 3-hydroxyleucine, 4-azaleucine, 5,5,5-trifluoroleucine, and so forth (Japanese Patent Publication No. 62-34397 and Japanese Patent Laid-open No. 8-70879), E. coli strains obtained by the genetic engineering method described in WO96/06926, E. coli H-9068 (Japanese Patent Laid-open No. 8-70879), and so forth.

The bacterium can be improved by enhancing expression of one or more genes involved in L-leucine biosynthesis. Examples of such genes include the genes of the leuABCD operon, a typical example of which is the mutant leuA gene coding for isopropyl malate synthase which has been mutated to be desensitized to feedback inhibition by L-leucine (U.S. Pat. No. 6,403,342). In addition, the bacterium can be improved by enhancing expression of one or more genes coding for proteins which increase export of L-amino acid from bacterial cells. Examples of such genes include b2682 and b2683 (the ygaZH genes) (EP 1239041 A2).

Examples of L-isoleucine-producing strains of coryneform bacteria include the coryneform bacterium of which brnE gene coding for a branched chain amino acid excretion protein is amplified (Japanese Patent Laid-open No. 2001-169788), the coryneform bacterium imparted with L-isoleucine-producing ability by protoplast fusion with an L-lysine-producing bacterium (Japanese Patent Laid-open No. 62-74293), the coryneform bacterium of which homoserine dehydrogenase is enhanced (Japanese Patent Laid-open No. 62-91193), the threonine hydroxamete resistant strain (Japanese Patent Laid-open No 62-195293), the α-ketomalonic acid resistant strain (Japanese Patent Laid-open No. 61-15695), and the methyl lysine resistant strain (Japanese Patent Laid-open No. 61-15696).

L-Histidine-Producing Bacteria

Examples of L-histidine-producing bacteria and parent strains which can be used to derive L-histidine-producing bacteria include, but are not limited to, Escherichia bacterial strains, such as E. coli strain 24 (VKPM B-5945, RU2003677), E. coli strain 80 (VKPM B-7270, Russian Patent No. 2119536), E. coli NRRL B-12116 to B-12121 (U.S. Pat. No. 4,388,405), E. coli H-9342 (FERM BP-6675), E. coli H-9343 (FERM BP-6676) (U.S. Pat. No. 6,344,347), E. coli H-9341 (FERM BP-6674) (EP 1085087 A), E. coli AI80/pFM201 (U.S. Pat. No. 6,258,554), and so forth.

Examples of L-histidine-producing bacteria and parent strains which can be used to derive L-histidine-producing bacteria also include strains in which the expression of one or more genes encoding L-histidine biosynthetic enzymes are enhanced. Examples of such genes include the ATP phosphoribosyltransferase gene (hisG), phosphoribosyl AMP cyclohydrolase gene (hisl), phosphoribosyl-ATP pyrophosphohydrolase gene (hisl), phosphoribosylformimino-5-aminoimidazole carboxamide ribotide isomerase gene (hisA), amidotransferase gene (hisH), histidinol phosphate aminotransferase gene (hisC), histidinol phosphatase gene (hisB), histidinol dehydrogenase gene (hisD), and so forth.

It is known that the L-histidine biosynthetic enzymes encoded by hisG and hisBHAFI are inhibited by L-histidine, and therefore the ability to produce L-histidine can also be efficiently enhanced by introducing a mutation which confers resistance to feedback inhibition into the gene coding for ATP phosphoribosyltransferase (hisG) (Russian Patent Nos. 2003677 and 2119536).

Specific examples of strains which are able to produce L-histidine include E. coli FERM-P 5038 and 5048 which have been transformed with a vector carrying a DNA encoding an L-histidine biosynthetic enzyme (Japanese Patent Laid-open No. 56-005099), E. coli strains transformed with a gene encoding a protein involved in amino acid export (EP 1016710 A), E. coli 80 strain which is resistant to sulfaguanidine, DL-1,2,4-triazole-3-alanine, and streptomycin (VKPM B-7270, Russian Patent No. 2119536), and so forth.

L-Glutamic Acid-Producing Bacteria

Examples of L-glutamic acid-producing bacteria and parent strains which can be used to derive L-glutamic acid-producing bacteria include, but are not limited to, Escherichia bacterial strains, such as E. coli VL334thrC⁺ (EP 1172433). E. coli VL334 (VKPM B-1641) is auxotrophic for L-isoleucine and L-threonine and contains mutant thrC and ilvA genes (U.S. Pat. No. 4,278,765). A wild-type allele of the thrC gene was transferred by general transduction using bacteriophage P1 grown on wild-type E. coli K12 (VKPM B-7) cells, resulting in the L-isoleucine auxotrophic L-glutamic acid-producing strain VL334thrC⁺ (VKPM B-8961).

Examples of L-glutamic acid-producing bacteria and parent strains which can be used to derive L-glutamic acid-producing bacteria also include, but are not limited to, strains in which expression of one or more genes encoding an L-glutamic acid biosynthetic enzyme is enhanced. Examples of such genes include the genes encoding glutamate dehydrogenase (gdhA), glutamine synthetase (glnA), glutamate synthetase (gltAB), isocitrate dehydrogenase (icdA), aconitate hydratase (acnA, acnB), citrate synthase (gltA), methyl citrate synthase (prpC), phosphoenolpyruvate carboxylase (ppc), pyruvate dehydrogenase (aceEF, 1pdA), pyruvate kinase (pykA, pykF), phosphoenolpyruvate synthase (ppsA), enolase (eno), phosphoglyceromutase (pgmA, pgmI), phosphoglycerate kinase (pgk), glyceraldehyde-3-phophate dehydrogenase (gapA), triose phosphate isomerase (tpiA), fructose bisphosphate aldolase (fbp), phosphofructokinase (pfkA, pfkB), glucose phosphate isomerase (pgi), and so forth. Among these enzymes, glutamate dehydrogenase, citrate synthase, phosphoenolpyruvate carboxylase, and methyl citrate synthase are preferred.

Examples of strains which have been modified so that expression of the citrate synthetase gene, the phosphoenolpyruvate carboxylase gene, and/or the glutamate dehydrogenase gene is enhanced include those disclosed in EP 1078989 A, EP 955368 A, and EP 952221 A.

Examples of L-glutamic acid-producing bacteria and parent strains which can be used to derive L-glutamic acid-producing bacteria also include strains in which the activity of one or more enzymes that catalyze one or more reactions which direct synthesis of one or more compounds other than L-glutamic acid, for example, by directing synthesis away from the biosynthetic pathway of L-glutamic acid, is reduced or eliminated. Examples of these enzymes include isocitrate lyase (aceA), α-ketoglutarate dehydrogenase (sucA), phosphotransacetylase (pta), acetate kinase (ack), acetohydroxy acid synthase (ilvG), acetolactate synthase (ilvI), formate acetyltransferase (pfl), lactate dehydrogenase (ldh), glutamate decarboxylase (gadAB), and so forth. Escherichia bacteria without α-ketoglutarate dehydrogenase activity or with reduced α-ketoglutarate dehydrogenase activity and methods to obtain such bacteria are described in U.S. Pat. Nos. 5,378,616 and 5,573,945.

Specifically, these strains include the following:

E. coli W3110sucA::Km^{r}

E. coli AJ12624 (FERM BP-3853)

E. coli AJ12628 (FERM BP-3854)

E. coli AJ12949 (FERM BP-4881)

E. coli W3110sucA::Km^{r} is obtained by disrupting the α-ketoglutarate dehydrogenase gene (hereinafter also referred to as the “sucA gene”) of E. coli W3110. This strain is completely deficient in α-ketoglutarate dehydrogenase.

Examples of coryneform bacteria with decreased α-ketoglutarate dehydrogenase activity include, for example, the following strains:

Brevibacterium lactofermentum L30-2 strain (Japanese Patent Laid-open No. 2006-340603)

Brevibacterium lactofermentum ΔS strain (WO95/34672)

Brevibacterium lactofermentum AJ12821 (FERM BP-4172, French Patent No. 9401748)

Brevibacterium flavum AJ12822 (FERM BP-4173, French Patent No. 9401748)

Corynebacterium glutamicum AJ12823 (FERM BP-4174, French Patent No. 9401748)

Corynebacterium glutamicum L30-2 strain (Japanese Patent Laid-open No. 2006-340603)

Other examples of L-glutamic acid-producing bacterium include Escherichia bacteria which are resistant to an aspartic acid antimetabolite. These strains can also be deficient in α-ketoglutarate dehydrogenase, and include, for example, E. coli AJ13199 (FERM BP-5807) (U.S. Pat. No. 5,908,768), FFRM P-12379, which additionally is decreased in an activity to decompose L-glutamic acid (U.S. Pat. No. 5,393,671); AJ13138 (FERM BP-5565) (U.S. Pat. No. 6,110,714), and so forth.

An example of an L-glutamic acid-producing bacterium which belongs to Pantoea ananatis is the Pantoea ananatis AJ13355 strain. This strain was isolated from soil in Iwata-shi, Shizuoka-ken, Japan, and was identified as being able to proliferate in a medium containing L-glutamic acid and a carbon source at a low pH. The Pantoea ananatis AJ13355 strain was deposited at the National Institute of Advanced Industrial Science and Technology, International Patent Organism Depositary (Tsukuba Central 6, 1-1, Higashi 1-Chome, Tsukuba-shi, Ibaraki-ken, 305-8566, Japan) on Feb. 19, 1998 and assigned an accession number of FERM P-16644. It was then converted to an international deposit under the provisions of Budapest Treaty on Jan. 11, 1999 and assigned an accession number of FERM BP-6614. This strain was originally identified as Enterobacter agglomerans when it was isolated, and deposited as Enterobacter agglomerans AJ13355. However, it was recently re-classified as Pantoea ananatis on the basis of nucleotide sequencing of 16S rRNA and so forth.

Furthermore, examples of an L-glutamic acid-producing bacterium of Pantoea ananatis also include Pantoea bacteria deficient in α-ketoglutarate dehydrogenase (αKGDH) activity or having reduced αKGDH activity. Examples of such a strain include AJ13356 (U.S. Pat. No. 6,331,419), which was derived by deleting the αKGDH-E1 subunit gene (sucA) in AJ13355, and the SC17sucA strain (U.S. Pat. No. 6,596,517), which also does not have the sucA gene, and was selected from AJ13355 for its low phlegm production properties. The AJ13356 strain was deposited at the National Institute of Bioscience and Human-Technology, Agency of Industrial Science and Technology (currently, the independent administrative agency, National Institute of Advanced Industrial Science and Technology, International Patent Organism Depositary (Tsukuba Central 6, 1-1, Higashi 1-Chome, Tsukuba-shi, Ibaraki-ken, Japan, postal code: 305-8566)) on Feb. 19, 1998, and assigned an accession number of FERM P-16645. Then, the deposit was converted into an international deposit under the provisions of the Budapest Treaty on Jan. 11, 1999, and assigned an accession number of FERM BP-6616. Although the AJ13355 and AJ13356 strains were deposited at the aforementioned depository as Enterobacter agglomerans, they are referred to as Pantoea ananatis in this specification. The SC17sucA strain was assigned the private number of AJ417, and deposited at the National Institute of Advanced Industrial Science and Technology, International Patent Organism Depositary on Feb. 26, 2004, under an accession number of FERM BP-08646.

Examples of L-glutamic acid-producing Pantoea ananatis bacteria further include SC17sucA/RSFCPG+pSTVCB, AJ13601, NP106, and NA1 strains. The SC17sucA/RSFCPG+pSTVCB strain was obtained by introducing the plasmid RSFCPG containing the citrate synthase gene (gltA), phosphoenolpyruvate carboxylase gene (ppsA), and glutamate dehydrogenase gene (gdhA) derived from Escherichia coli, and the plasmid pSTVCB containing the citrate synthase gene (gltA) derived from Brevibacterium lactofermentum, into the SC17sucA strain. The AJ13601 strain was selected from the SC17sucA/RSFCPG+pSTVCB strain for its resistance to high concentration of L-glutamic acid at a low pH. Furthermore, the NP106 strain was derived from the AJ13601 strain by eliminating the RSFCPG+pSTVCB plasmid. The AJ13601 strain was deposited at the National Institute of Advanced Industrial Science and Technology, International Patent Organism Depositary (Tsukuba Central 6, 1-1, Higashi 1-Chome, Tsukuba-shi, Ibaraki-ken, Japan, postal code: 305-8566) on Aug. 18, 1999, and assigned accession number FERM P-17516. Then, the deposit was converted into an international deposit under the provisions of the Budapest Treaty on Jul. 6, 2000, and assigned an accession number FERM BP-7207.

Furthermore, the ability to produce L-glutamic acid can also be imparted to coryneform bacteria by a method of amplifying the yggB gene coding for the mechanosensitive channel (WO2006/070944), and a method of introducing a mutant yggB gene in which a mutation is introduced into the coding region. The yggB gene is located at the nucleotide numbers 1,337,692 to 1,336,091 (complementary strand) of the genome sequence of Corynebacterium glutamicum ATCC 13032 strain registered with GenBank Accession No. NC_{—}003450, and coding for a membrane protein also called NCg11221 and registered with GenBank accession No. NP_{—}600492.

Examples of other methods for imparting or enhancing L-glutamic acid-producing ability also include a method of imparting resistance to an organic acid analogue, a respiratory chain inhibitor, etc., and a method of imparting sensitivity to a cell wall synthesis inhibitor. Examples of such methods include the method of imparting resistance to monofluoroacetic acid (Japanese Patent Laid-open No. 50-113209), the method of imparting resistance to adenine or thymine (Japanese Patent Laid-open No. 57-065198), the method of attenuating urease (Japanese Patent Laid-open No. 52-038088), the method of imparting resistance to malonic acid (Japanese Patent Laid-open No. 52-038088), the method of imparting resistance to benzopyrones or naphthoquinones (Japanese Patent Laid-open No. 56-1889), the method of imparting resistance to HOQNO (Japanese Patent Laid-open No. 56-140895), the method of imparting resistance to α-ketomalonic acid (Japanese Patent Laid-open No. 57-2689), the method of imparting resistance to guanidine (Japanese Patent Laid-open No. 56-35981), the method of imparting sensitivity to penicillin (Japanese Patent Laid-open No. 4-88994), and so forth.

Specific examples of such resistant strains include the following strains:

Brevibacterium flavum AJ3949 (FERM BP-2632; Japanese Patent Laid-open No. 50-113209)

Corynebacterium glutamicum AJ11628 (FERM P-5736; Japanese Patent Laid-open No. 57-065198)

Brevibacterium flavum AJ11355 (FERM P-5007; Japanese Patent Laid-open No. 56-1889)

Corynebacterium glutamicum AJ11368 (FERM P-5020; Japanese Patent Laid-open No. 56-1889)

Brevibacterium flavum AJ11217 (FERM P-4318; Japanese Patent Laid-open No. 57-2689)

Corynebacterium glutamicum AJ11218 (FERM P-4319; Japanese Patent Laid-open No. 57-2689)

Brevibacterium flavum AJ11564 (FERM BP-5472; Japanese Patent Laid-open No. 56-140895)

Brevibacterium flavum AJ11439 (FERM BP-5136; Japanese Patent Laid-open No. 56-35981)

Corynebacterium glutamicum H7684 (FERM BP-3004; Japanese Patent Laid-open No. 04-88994)

Brevibacterium lactofermentum AJ11426 (FERM P-5123; Japanese Patent Laid-open No. 56-048890)

Corynebacterium glutamicum AJ11440 (FERM P-5137; Japanese Patent Laid-open No. 56-048890)

Brevibacterium lactofermentum AJ11796 (FERM P-6402; Japanese Patent Laid-open No. 58-158192)

L-Phenylalanine-Producing Bacteria

Examples of L-phenylalanine-producing bacteria and parent strains which can be used to derive L-phenylalanine-producing bacteria include, but are not limited to, Escherichia bacterial strains, such as E. coli AJ12739 (tyrA::Tn10, tyrR) (VKPM B-8197) which lacks chorismate mutase-prephenate dehydrogenase and the tyrosine repressor (WO03/044191), E. coli HW1089 (ATCC 55371) which contains the pheA34 gene coding for chorismate mutase-prephenate dehydratase which has been mutated to be desensitized to feedback inhibition (U.S. Pat. No. 5,354,672), E. coli MWEC101-b (KR8903681), E. coli NRRL B-12141, NRRL B-12145, NRRL B-12146, and NRRL B-12147 (U.S. Pat. No. 4,407,952). Also, the following strains can be used to derive L-phenylalanine-producing bacteria: E. coli K-12 [W3110(tyrA)/pPHAB (FERM BP-3566) which contains genes coding for chorismate mutase-prephenate dehydratase which has been mutated to be desensitized to feedback inhibition, E. coli K-12 [W3110(tyrA)/pPHAD] (FERM BP-12659), E. coli K-12 [W3110(tyrA)/pPHATerm] (FERM BP-12662), and E. coli K-12 [W3110(tyrA)/pBR-aroG4, pACMAB] (also known as AJ12604 (FERM BP-3579) (EP 488424 B1). Furthermore, Escherichia L-phenylalanine-producing bacteria with enhanced activity of the protein encoded by the yedA gene or the yddG gene can also be used (U.S. Patent Published Applications Nos. 2003/0148473 and 2003/0157667, WO03/044192).

As phenylalanine-producing coryneform bacteria, the Cornebacterium glutamicum BPS-13 (FERM BP-1777), K77 (FERM BP-2062), and K78 (FERM BP-2063) (EP 331145 A, Japanese Patent Laid-open No. 02-303495), of which phosphoenolpyruvate carboxylase or pyruvate kinase activity is reduced, tyrosine-auxotrophic strain (Japanese Patent Laid-open No. 05-049489), and so forth can be used.

A bacterium which efficiently produces phenylalanine can also be obtained by modifying a bacterium so that the bacterium incorporates by-products, for example, by increasing the expression amount of the L-tryptophan uptake gene, tnaB or mtr, or the L-tyrosine uptake gene, tyrP (European Patent No. 1484410).

L-Tryptophan-Producing Bacteria

Examples of L-tryptophan-producing bacteria and parent strains which can be used to derive L-tryptophan-producing bacteria include, but are not limited to, Escherichia bacterial strains, such as E. coli JP4735/pMU3028 (DSM10122) and E. coli JP6015/pMU91 (DSM10123) which lack tryptophanyl-tRNA synthetase encoded by a mutant trpS gene (U.S. Pat. No. 5,756,345), E. coli SV164 (pGH5) which contains the serA allele encoding phosphoglycerate dehydrogenase and the trpE allele encoding anthranilate synthase, which are desensitized to feedback inhibition by serine and tryptophan, respectively (U.S. Pat. No. 6,180,373), E. coli AGX17 (pGX44) (NRRL B-12263), and E. coli AGX6(pGX50)aroP (NRRL B-12264) which lack tryptophanase (U.S. Pat. No. 4,371,614), and E. coli AGX17/pGX50, pACKG4-pps in which phosphoenolpyruvate-producing ability is enhanced (WO97/08333, U.S. Pat. No. 6,319,696). L-Tryptophan-producing bacteria belonging to the genus Escherichia with enhanced activity of the protein encoded by the yedA gene or the yddG gene can also be used (U.S. Patent Published Application Nos. 2003/0148473 and 2003/0157667).

Examples of L-tryptophan-producing bacteria and parent strains which can be used to derive L-tryptophan-producing bacteria also include strains in which one or more activities of the following enzymes are enhanced: anthranilate synthase (trpE), phosphoglycerate dehydrogenase (serA), 3-deoxy-D-arabinoheptulosonate-7-phosphate synthase (aroG), 3-dehydroquinate synthase (aroB), shikimate dehydrogenase (aroE), shikimate kinase (aroL), 5-enolpyruvylshikimate-3-phosphate synthase (aroA), chorismate synthase (aroC), prephenate dehydratase, chorismate mutase, and tryptophan synthase (trpAB). Prephenate dehydratase and chorismate mutase are encoded by the pheA gene as a bifunctional enzyme (chorismate mutase/prephenate dehydratase, CM/PDH). Among these enzymes, phosphoglycerate dehydrogenase, 3-deoxy-D-arabinoheptulosonate-7-phosphate synthase, 3-dehydroquinate synthase, shikimate dehydratase, shikimate kinase, 5-enolpyruvylshikimate-3-phosphate synthase, chorismate synthase, prephenate dehydratase, and chorismate mutase-prephenate dehydratase are particularly preferred. Anthranilate synthase and phosphoglycerate dehydrogenase both suffer from feedback inhibition by L-tryptophan and L-serine, and therefore a mutation desensitizing feedback inhibition can be introduced into the genes encoding these enzymes. Specific examples of strains having such a mutation include E. coli SV164 having a desensitized type anthranilate synthase and a transformant strain obtained by introducing pGH5 (WO94/08031) containing a mutant serA gene coding for phosphoglycerate dehydrogenase desensitized to feedback inhibition into E. coli SV164.

Examples of L-tryptophan-producing bacteria and parent strains which can be used to derive L-tryptophan-producing bacteria also include strains which have been transformed with the tryptophan operon, which contains a gene encoding inhibition-desensitized anthranilate synthase (Japanese Patent Laid-open Nos. 57-71397, 62-244382, U.S. Pat. No. 4,371,614). Moreover, L-tryptophan-producing ability can be imparted by enhancing expression of a gene which encodes tryptophan synthase in the tryptophan operon (trpBA). Tryptophan synthase includes both α and β subunits, which are encoded by trpA and trpB, respectively. In addition, L-tryptophan-producing ability can be improved by enhancing expression of the isocitrate lyase-malate synthase operon (WO2005/103275).

As coryneform bacteria, Corynebacterium glutamicum AJ12118 (FERM BP-478, Japanese Patent No. 01681002), which is resistant to sulfaguanidine, the coryneform bacterium introduced with the tryptophan operon (Japanese Patent Laid-open No. 63-240794), and the coryneform bacterium introduced with a gene coding for shikimate kinase derived from a coryneform bacterium (Japanese Patent Laid-open No. 01-994749) can be used.

L-Proline-Producing Bacteria

Examples of L-proline-producing bacteria and parent strains which can be used to derive L-proline-producing bacteria include, but are not limited to, Escherichia bacterial strains, such as E. coli 702ilvA (VKPM B-8012) which lacks the ilvA gene and can produce L-proline (EP 1172433 A).

The bacterium can be improved by enhancing expression of one or more genes involved in L-proline biosynthesis. Examples of genes preferred for L-proline-producing bacteria include the proB gene coding for glutamate kinase which is desensitized to feedback inhibition by L-proline (DE Patent No. 3127361). In addition, the bacterium can be improved by enhancing expression of one or more genes coding for proteins responsible for secretion of L-amino acids from the bacterial cell. Examples of such genes are b2682 and b2683 genes (ygaZH genes) (EP 1239041 A2).

Escherichia bacteria which produce L-proline include the following E. coli strains: NRRL B-12403 and NRRL B-12404 (GB Patent No. 2075056), VKPM B-8012 (Russian Patent Application No. 2000124295), plasmid mutants described in DE Patent No. 3127361, plasmid mutants described by Bloom F. R. et al (The 15th Miami Winter Symposium, 1983, p. 34), and so forth.

L-Arginine-Producing Bacteria

Examples of L-arginine-producing bacteria and parent strains which can be used to derive L-arginine-producing bacteria include, but are not limited to, Escherichia bacterial strains, such as E. coli strain 237 (VKPM B-7925) (U.S. Patent Published Application No. 2002/058315 A1) and its derivative strains harboring mutant N-acetylglutamate synthase (Russian Patent Application No. 2001112869), E. coli strain 382 (VKPM B-7926) (EP 1170358 A1), and an arginine-producing strain transformed with an argA gene encoding N-acetylglutamate synthetase (EP 1170361 A1).

Examples of L-arginine-producing bacteria and parent strains which can be used to derive L-arginine-producing bacteria also include strains in which the expression of one or more genes encoding an L-arginine biosynthetic enzyme is enhanced. Examples of such genes include the N-acetylglutamyl phosphate reductase gene (argC), ornithine acetyl transferase gene (argJ), N-acetylglutamate kinase gene (argB), acetylornithine transaminase gene (argD), ornithine carbamoyl transferase gene (argF), argininosuccinic acid synthetase gene (argG), argininosuccinic acid lyase gene (argH), and carbamoyl phosphate synthetase gene (carAB).

L-Valine-Producing Bacteria

Examples of L-valine-producing bacteria and parent strains which can be used to derive L-valine-producing bacteria include, but are not limited to, strains which have been modified to overexpress the ilvGMEDA operon (U.S. Pat. No. 5,998,178). It is desirable to remove the region in the ilvGMEDA operon, which is required for attenuation, so that expression of the operon is not attenuated by the produced L-valine. Furthermore, the ilvA gene in the operon is desirably disrupted so that threonine deaminase activity is decreased.

Examples of L-valine-producing bacteria and parent strains which can be used to derive L-valine-producing bacteria also include mutants having amino-acyl t-RNA synthetase mutations (U.S. Pat. No. 5,658,766). An example is E. coli VL1970, which has a mutation in the ileS gene encoding isoleucine tRNA synthetase. E. coli VL1970 was deposited at the Russian National Collection of Industrial Microorganisms (VKPM) (1 Dorozhny proezd., 1 Moscow 117545, Russia) on Jun. 24, 1988 under the accession number VKPM B-4411.

Furthermore, mutant strains which require lipoic acid for growth and/or lack Ht ATPase (WO96/06926) are also effective to derive L-valine-producing bacteria.

Examples of L-valine-producing bacteria of coryneform bacteria include, for example, strains modified so that expression of a gene encoding an L-valine biosynthetic enzyme is enhanced. Examples of the L-valine biosynthesis enzyme include enzymes encoded by genes present on the ilvBNC operon, that is, acetohydroxy acid synthetase encoded by ilvBN and isomero-reductase encoded by ilvC (WO00/50624). Since the ilvBNC operon is subject to expression regulation by L-valine and/or L-isoleucine and/or L-leucine, it is desirable to eliminate attenuation to avoid expression suppression by L-valine that is produced.

Impartation of L-valine-producing ability to coryneform bacteria may be performed by decreasing or eliminating activity of at least one kind of enzyme which is involved in a metabolic pathway that decreases L-valine production. For example, decrease of the activity of threonine dehydratase involved in the L-leucine synthesis, or activity of an enzyme that involved in D-panthothenate synthesis is contemplated (WO00/50624).

Examples of methods for imparting L-valine-producing ability also include imparting resistance to an amino acid analogue or the like.

Examples include, for example, mutant strains which are auxotrophic for L-isoleucine and L-methionine, and resistant to D-ribose, purine ribonucleoside or pyrimidine ribonucleoside, and have an ability to produce L-valine (FERM P-1841, FERM P-29, Japanese Patent Publication No. 53-025034), mutant strains resistant to polyketides (FERM P-1763, FERM P-1764, Japanese Patent Publication No. 06-065314), and mutant strains resistant to L-valine in a medium containing acetic acid as the sole carbon source and sensitive to pyruvic acid analogues (fluoropyruvic acid etc.) in a medium containing glucose as the sole carbon source (FERM BP-3006, BP-3007, Japanese Patent No. 3006929).

L-Isoleucine-Producing Bacteria

Examples of L-isoleucine producing bacteria and parent strains which can be used to derive L-isoleucine-producing bacteria include, but are not limited to, mutants which are resistant to 6-dimethylaminopurine (Japanese Patent Laid-open No. 5-304969), mutants which are resistant to isoleucine analogues such as thiaisoleucine and isoleucine hydroxamate, and mutants which are additionally resistant to DL-ethionine and/or arginine hydroxamate (Japanese Patent Laid-open No. 5-130882). In addition, recombinant strains transformed with genes encoding proteins involved in L-isoleucine biosynthesis, such as threonine deaminase and acetohydroxate synthase, are also effective to derive L-isoleucine-producing bacteria (Japanese Patent Laid-open No. 2-458, FR 0356739, and U.S. Pat. No. 5,998,178).

Examples of L-isoleucine-producing strains of coryneform bacteria include the coryneform bacterium of which brnE gene coding for a branched chain amino acid excretion protein is amplified (Japanese Patent Laid-open No. 2001-169788), the coryneform bacterium imparted with L-isoleucine-producing ability by protoplast fusion with an L-lysine-producing bacterium (Japanese Patent Laid-open No. 62-74293), the coryneform bacterium of which homoserine dehydrogenase is enhanced (Japanese Patent Laid-open No. 62-91193), the threonine hydroxamete resistant strain (Japanese Patent Laid-open No 62-195293), the α-ketomalonic acid resistant strain (Japanese Patent Laid-open No. 61-15695), and the methyl lysine resistant strain (Japanese Patent Laid-open No. 61-15696).

L-Methionine-Producing Bacteria

Examples of L-methionine-producing bacteria and parent strains for deriving L-methionine producing bacteria include, but are not limited to, L-threonine-auxotrophic mutant strain and norleucine-resistant mutant strain (Japanese Patent Laid-open No. 2000-139471). Furthermore, a methionine repressor-deficient strain and recombinant strains transformed with genes encoding proteins involved in L-methionine biosynthesis such as homoserine transsuccinylase and cystathionine γ-synthase (Japanese Patent Laid-open No. 2000-139471) can also be used as parent strains.

When the aforementioned L-amino acid-producing bacteria are bred by genetic recombination, the genes to be used are not limited to genes having the genetic information described above or genes having known sequences, but also include genes having conservative mutations, such as homologues or artificially modified genes, can also be used so long as the functions of the encoded proteins are not degraded. That is, they may be genes encoding a known amino acid sequence containing one or more substitutions, deletions, insertions, additions or the like of one or several amino acid residues at one or several positions.

Although the number of the “one or several” amino acid residues referred to herein may differ depending on the position in the three-dimensional structure or the types of amino acid residues of the protein, specifically, it may be 1 to 20, 1 to 10, or 1 to 5. The conservative mutation is a mutation wherein substitution takes place mutually among Phe, Trp, and Tyr, if the substitution site is an aromatic amino acid; among Leu, Ile and Val, if it is a hydrophobic amino acid; between Gln and Asn, if it is a polar amino acid; among Lys, Arg and His, if it is a basic amino acid; between Asp and Glu, if it is an acidic amino acid; and between Ser and Thr, if it is an amino acid having a hydroxyl group. The conservative mutation is typically a conservative substitution, and substitutions considered conservative substitutions include, specifically, substitution of Ser or Thr for Ala, substitution of Gln, His or Lys for Arg, substitution of Glu, Gln, Lys, His or Asp for Asn, substitution of Asn, Glu or Gln for Asp, substitution of Ser or Ala for Cys, substitution of Asn, Glu, Lys, His, Asp or Arg for Gln, substitution of Gly, Asn, Gln, Lys or Asp for Glu, substitution of Pro for Gly, substitution of Asn, Lys, Gln, Arg or Tyr for His, substitution of Leu, Met, Val or Phe for Ile, substitution of Ile, Met, Val or Phe for Leu, substitution of Asn, Glu, Gln, His or Arg for Lys, substitution of Ile, Leu, Val or Phe for Met, substitution of Trp, Tyr, Met, Ile or Leu for Phe, substitution of Thr or Ala for Ser, substitution of Ser or Ala for Thr, substitution of Phe or Tyr for Trp, substitution of His, Phe or Trp for Tyr, and substitution of Met, Ile or Leu for Val. The aforementioned amino acid substitutions, deletions, insertions, additions, inversions or the like may be a result of a naturally-occurring mutation or a variation due to an individual difference or difference of species of a microorganism from which the genes are derived (mutant or variant). Such genes can be obtained by, for example, modifying a known nucleotide sequence of a gene by site-specific mutagenesis so that the amino acid residues at the specific sites of the encoded protein include substitutions, deletions, insertions, or additions of amino acid residues.

Furthermore, such genes having conservative mutation(s) as described above may encode a protein having a homology of 80% or more, 90% or more, 95% or more, or 97% or more, to the entire encoded amino acid sequence and having a function equivalent to that of the wild-type protein.

Moreover, codons in the gene sequences may be replaced with other codons which are easily used in the host into which the genes are introduced.

The genes having conservative mutation(s) may be obtained by methods usually used in mutagenesis treatments such as treatments with mutagenesis agents.

Furthermore, the genes may be a DNA which can hybridize with a complementary sequence of a known gene sequence or a probe which can be prepared from the complementary sequence under stringent conditions and encodes a protein having a function equivalent to that of the known gene product. The “stringent conditions” referred to here can be conditions under which a so-called specific hybrid is formed, and a non-specific hybrid is not formed. Examples of the stringent conditions include those under which highly homologous DNAs hybridize to each other, for example, DNAs not less than 80% homologous, not less than 90% homologous, not less than 95% homologous, or not less than 97% homologous, hybridize to each other, and DNAs less homologous than the above do not hybridize to each other, or conditions of washing once, preferably 2 or 3 times, at a salt concentration and temperature corresponding to washing typical of Southern hybridization, i.e., 1×SSC, 0.1% SDS at 60° C., 0.1×SSC, 0.1% SDS at 60° C., or 0.1×SSC, 0.1% SDS at 68° C.

As the probe, a part of the sequence which is complementary to the gene can also be used. Such a probe can be prepared by PCR using oligonucleotides prepared on the basis of the known gene sequence as primers and a DNA fragment containing the nucleotide sequences as a template. For example, when a DNA fragment having a length of about 300 bp is used as the probe, the washing conditions of hybridization may be 50° C., 2×SSC and 0.1% SDS.

<4> Method for Producing L-Amino Acid

The method for producing an L-amino acid can be a method for producing an L-amino acid comprising producing a fatty acid by the method for producing a fatty acid of the present invention, culturing a bacterium having an L-amino acid-producing ability in a medium containing the fatty acid to produce and accumulate an L-amino acid in the culture, and collecting the L-amino acid from the culture.

The fatty acid contained in the medium is usually used as a carbon source for the L-amino acid fermentation. The expression “as a carbon source” mentioned above means that the fatty acid can substantially contribute as a supply source of carbon constituting cell components and L-amino acids in proliferation of the bacterium and L-amino acid production.

For the method of the present invention, batch culture, fed-batch culture and continuous culture may be used. The fatty acid may be contained in starting medium or feed medium, or may be contained in these both.

The fed-batch culture refers to a culture method in which a medium is continuously or intermittently fed into a culture vessel, and the medium is not extracted until the end of culture. The continuous culture means a method in which a medium is continuously or intermittently fed into a culture vessel, and the medium is extracted from the vessel (usually in a volume equivalent to the volume of fed medium) at the same time. The starting medium means the medium used in batch culture in the fed-batch culture or continuous culture before feeding the feed medium (medium used at the time of the start of the culture), and feed medium means a medium which is supplied to a fermentation tank in the fed-batch culture or continuous culture. The batch culture means a method in which fresh medium is prepared for every culture, and a strain is inoculated into the medium, which medium is not changed until harvest.

The fatty acid to be used may be used at any concentration so long as the concentration is suitable for producing an L-amino acid. It is usually desirable to add the fatty acid to the medium at a concentration of about 0.01 to 10 w/v %, about 0.02 to 5 w/v %, or about 0.05 to 2 w/v %. As the carbon source, the fatty acid alone may be used, or it may also be used in combination with other carbon sources such as glucose, fructose, sucrose, blackstrap molasses, and starch hydrolysate. In the latter case, although the fatty acid and other carbon sources may be mixed at an arbitrary ratio, it is desirable that the ratio of the fatty acid in the carbon source is 10% by weight or more, 50% by weight or more, or 70% by weight or more. Preferred as the other carbon sources are saccharides such as glucose, fructose, sucrose, lactose, galactose, blackstrap molasses, starch hydrolysate, and a sugar solution obtained by hydrolysis of biomass, alcohols such as ethanol and glycerol, and organic acids such as fumaric acid, citric acid, and succinic acid. In the present invention, a fatty acid exists in the residue produced by the Byg-Dye method or the like, and glycerol existing in the supernatant may also be used as the carbon source.

As the medium to be used, media conventionally used in the production of L-amino acids by fermentation using microorganisms can be used, provided that the medium contains a fatty acid. That is, conventional media containing, besides a carbon source, a nitrogen source, inorganic ions, and optionally other organic components as required may be used. As the nitrogen source, inorganic ammonium salts such as ammonium sulfate, ammonium chloride, ammonium phosphate, ammonium acetate, and urea, nitrates, organic nitrogen such as soybean hydrolysate, ammonia gas, aqueous ammonia, and so forth may be used. Furthermore, peptone, yeast extract, meat extract, malt extract, corn steep liquor, soybean hydrolysate and so forth can also be utilized. The medium may contain one or more types of these nitrogen sources. These nitrogen sources can also be used for both the starting medium and the feed medium. Furthermore, the same nitrogen source can be used for both the starting medium and the feed medium, or the nitrogen source of the feed medium may be different from that of the starting medium.

The medium can contain a phosphoric acid source and a sulfur source in addition to the carbon source and the nitrogen source. As the phosphoric acid source, potassium dihydrogenphosphate, dipotassium hydrogenphosphate, phosphate polymers such as pyrophosphoric acid and so forth can be utilized. Although the sulfur source may be any substance containing sulfur atoms, sulfuric acid salts such as sulfates, thiosulfates and sulfites, and sulfur-containing amino acids such as cysteine, cystine and glutathione are desirable, and ammonium sulfate is especially desirable.

Furthermore, the medium may contain a growth promoting factor (nutrient having a growth promoting effect) in addition to the aforementioned components. As the growth promoting factor, trace metals, amino acids, vitamins, nucleic acids as well as peptone, casamino acid, yeast extract, soybean protein degradation product and so forth containing the foregoing substances can be used. Examples of the trace metals include iron, manganese, magnesium, calcium and so forth. Examples of the vitamins include vitamin B₁, vitamin B₂, vitamin B₆, nicotinic acid, nicotinamide, vitamin B₁₂ and so forth. These growth promoting factors may be contained in the starting medium or the feed medium.

Furthermore, when an auxotrophic mutant that requires an amino acid or the like for growth thereof is used, it is preferable to supplement a required nutrient to the medium. In particular, since the L-lysine biosynthetic pathway is enhanced and L-lysine degrading ability is often attenuated in L-lysine-producing bacteria that can be used for the present invention as described below, one or more types of substances selected from L-threonine, L-homoserine, L-isoleucine and L-methionine are preferably added. The starting medium and the feed medium may have the same or different medium composition. Furthermore, the starting medium and the feed medium may have the same or different sulfur concentration. Furthermore, when the feed medium is fed at multiple stages, the compositions of the feed media fed at the stages may be the same or different.

In addition, the medium may be either a natural medium or synthetic medium, so long as it contains a carbon source, a nitrogen source, and other components as required.

The culture can be performed for 1 to 7 days under aerobic conditions. The culture temperature is 20 to 45° C., 24 to 45° C., or 33 to 42° C. The culture can be performed as aeration culture, with controlling the oxygen concentration to be about 5 to 50%, for example about 10%, of the saturation concentration. Furthermore, pH can be controlled to be 5 to 9 during the culture. For adjusting pH, inorganic or organic acidic or alkaline substances, such as calcium carbonate, ammonia gas, and aqueous ammonia, can be used.

If culture is performed under such conditions as described above preferably for about 10 to 120 hours, a marked amount of L-amino acid is accumulated in the culture medium. Although the concentration of L-amino acid accumulated is not limited so long as it enables isolation and collection of the L-amino acid from the medium or cells, it can be 1 g/L or higher, 50 g/L or higher, or 100 g/L or higher.

When a basic amino acid such as L-lysine is produced, the production may be performed by a method in which fermentation is performed by controlling pH of the medium during culture to be 6.5 to 9.0 and pH of the medium at the end of the culture to be 7.2 to 9.0 and controlling the pressure in the fermentation tank to be positive during the culture, or by supplying carbon dioxide gas or a mixed gas containing carbon dioxide gas to the medium to provide a culture period where the medium contains 2 g/L 20 mM or more of bicarbonate ions and/or carbonate ions, so that these bicarbonate ions and/or carbonate ions serve as counter ions of cations mainly consisting of a basic amino acid, and the objective basic amino acid is then collected (Japanese Patent Laid-open No. 2002-65287, U.S. Patent Published Application No. 2002/0025564, EP 1813677 A).

Further, in L-glutamic acid fermentation, the culture can be performed with precipitating L-glutamic acid in the medium by using a liquid medium adjusted to have a condition under which L-glutamic acid is precipitated. The condition under which L-glutamic acid is precipitated is, for example, pH 5.0 to 4.0, pH 4.5 to 4.0, pH 4.3 to 4.0, or pH 4.0 (EP 1078989 A).

Collection of the L-amino acid from the culture medium can usually be attained by a combination of known methods such as an ion exchange resin method and precipitation method. When the L-amino acid accumulates in the cells, the cells can be disrupted with, for example, supersonic waves or the like, and the L-amino acid can be collected by the ion exchange resin method or the like from the supernatant obtained by removing the cells from the cell-disrupted suspension by centrifugation. The L-amino acid to be collected may be a free L-amino acid, or may be a salt such as sulfate, hydrochloride, carbonate, ammonium salt, sodium salt, and potassium salt.

The L-amino acid collected according to the present invention may contain bacterial cells, medium components, moisture, and by-product metabolites of the bacterium in addition to the objective L-amino acid. Purity of the collected L-amino acid is 50% or higher, 85% or higher, or 95% or higher (Japanese Patent No. 1214636, U.S. Pat. Nos. 5,431,933, 4,956,471, 4,777,051, 4,946,654, 5,840,358, 6,238,714, U.S. Patent Published Application No. 2005/0025878).

### EXAMPLES

Hereafter, the present invention will be explained more specifically with reference to the following non-limiting examples. In the examples, Chlorella kessleri 11H (UTEX 263), Chlorella Kessleri UTEX 398, Chlorella sorokiniana UTEX 1230, Scenedesmus dimorphus UTEX 417, Scenedesmus obliquus UTEX B2630, Nannochloris sp. UTEX LB1999, Nannochloris oculata UTEX LB1998, Neochloris oleoabundans UTEX 1185, and Dunaliella tertiolecta UTEX LB999 strains obtained from The University of Texas at Austin, The Culture Collection of Algae (UTEX) (1 University Station A6700, Austin, Tex. 78712-0183, USA) were used.

### Example 1

### Culture of Microalga Strain, Chlorella Kessleri 11 h, in Medium Bottle

The Chlorella kessleri 11 h strain was cultured at 30° C. and a light intensity of 7,000 luxes (culture apparatus CL-301, TOMY) for 7 days in 800 mL of the 0.2× Gamborg's B5 medium (NIHON PHARMACEUTICAL) contained in a 1000 mL-volume medium bottle with blowing 400 mL/minute of a mixed gas of air and 3% CO₂ into the medium, and the resultant culture fluid was used as a preculture. As the light source, white light from a fluorescent lamp was used. The preculture in a volume of 16 mL was added to 800 mL of the 0.2× Gamborg's B5 medium contained in a 1000 mL-volume medium bottle, and culture was performed at a culture temperature of 30° C. and a light intensity of 7,000 luxes for 14 days with blowing 400 mL/minute of a mixed gas of air and 3% CO₂ into the medium.

0.2× Gamborg's B5 Medium:

| | | |
|---|---|---|
| KNO₃ | 500 | mg/L |
| MgSO₄•7H₂O | 50 | mg/L |
| NaH₂PO₄•H₂O | 30 | mg/L |
| CaCl₂•2H₂O | 30 | mg/L |
| (NH₄)₂SO₄ | 26.8 | mg/L |
| Na₂-EDTA | 7.46 | mg/L |
| FeSO₄•7H₂O | 5.56 | mg/L |
| MnSO₄•H₂O | 2 | mg/L |
| H₃BO₃ | 0.6 | mg/L |
| ZnSO₄•7H₂O | 0.4 | mg/L |
| KI | 0.15 | mg/L |
| Na₂MoO₂•2H₂O | 0.05 | mg/L |
| CuSO₄•5H₂O | 0.005 | mg/L |
| CoCl₂•6H₂O | 0.005 | mg/L |

The medium was sterilized by autoclaving at 120° C. for 15 minutes.

### Example 2

### Examination of Temperature Condition for Reaction of Alga Culture after Addition of Alcohol

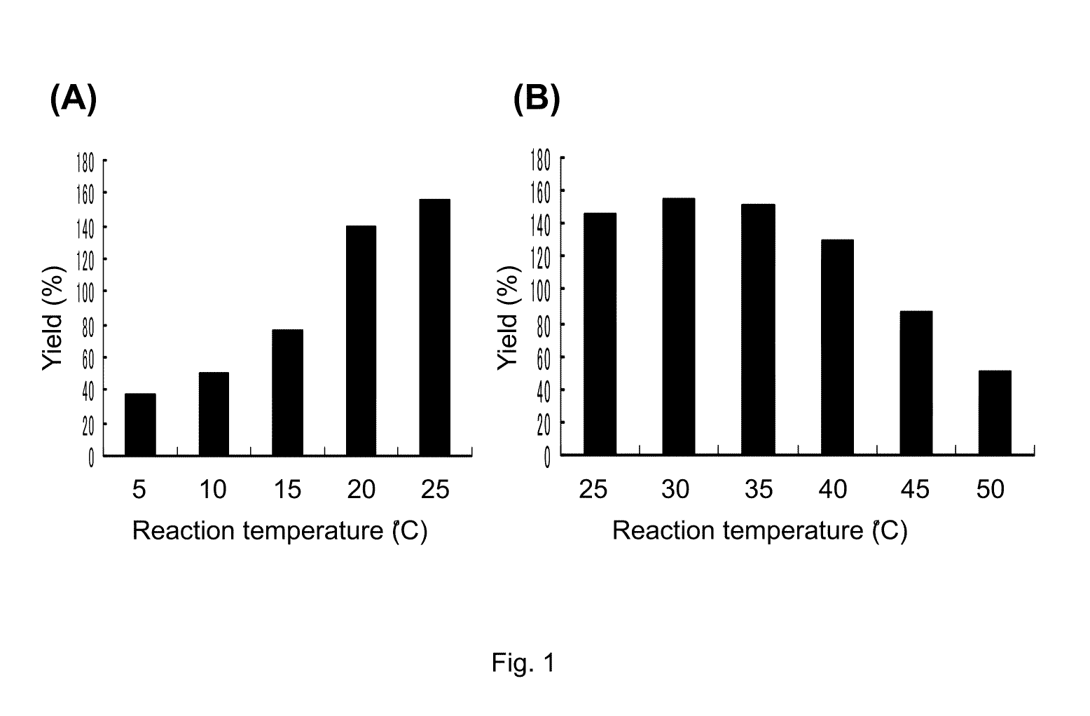

The culture fluid obtained in Example 1 was centrifuged, and sterilized water was added to the obtained precipitates to prepare a 1× suspension. The suspension was put into 1.5 ml-volume Eppendorf tubes in a volume of 1 ml each, and centrifuged again, and 200 μL each of a 30% aqueous solution of methanol was added to the obtained precipitates to obtain suspensions. The suspensions were incubated at temperatures of 5° C., 10° C., 15° C., 20° C., 25° C., 30° C., 35° C., 40° C., 45° C., and 50° C. for 2 hours with rotation shaking at 1,000 rpm to allow the transesterification reaction. Then, lipids were extracted from the obtained samples with an organic solvent according to the Bligh-Dyer method, and fatty acid methyl esters were measured. The measurement results are shown in FIG. 1. At low temperatures of 5° C., 10° C., and 15° C., and high temperatures of 45° C. and 50° C., the fatty acid ester yield tended to become low. In contrast, under the relatively mild conditions of 20° C., 25° C., 30° C., 35° C., 40° C., and 45° C., high fatty acid ester yields were confirmed.

### Example 3

### Examination of Methanol Concentration for Reaction of Alga Culture after Addition of Alcohol

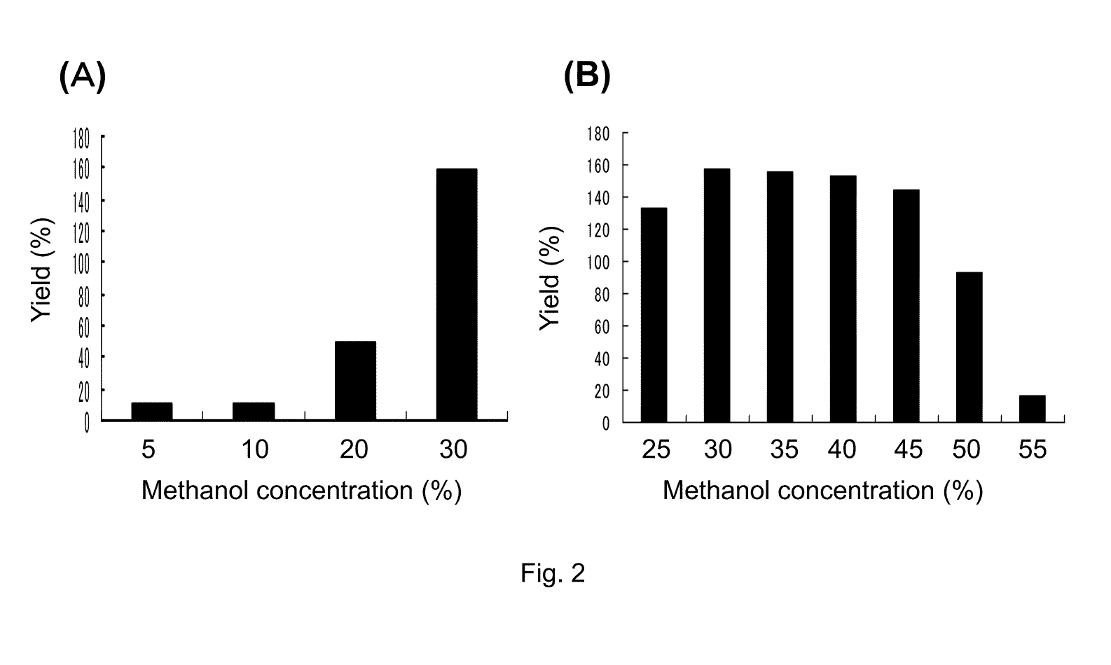

The culture fluid obtained in Example 1 was centrifuged, and sterilized water was added to the obtained precipitates to prepare a 1× suspension. The suspension was put into 1.5 ml-volume Eppendorf tubes in a volume of 1 ml each, and centrifuged again, and 200 μL each of aqueous solutions of methanol at methanol concentrations of 5%, 10%, 20%, 25%, 30%, 35%, 40%, 45%, 50%, and 55% were added to the obtained precipitates to obtain suspensions. The suspensions were incubated at 35° C. for 2 hours with rotation shaking at 1,000 rpm to allow the transesterification reaction. Then, lipids were extracted from the obtained samples with an organic solvent according to the Bligh-Dyer method, and fatty acid methyl esters were measured. The measurement results are shown in FIG. 2. With methanol solutions of low concentrations of 5%, 10%, and 20%, and methanol solutions of high concentrations of 50% and 55%, the fatty acid ester yield tended to become low. In contrast, with the added methanol concentrations of 25%, 30%, 35%, 40%, and 45%, high fatty acid ester yields were confirmed.

### Example 4

### Examination of pH Condition for Reaction of Alga Culture after Addition of Alcohol

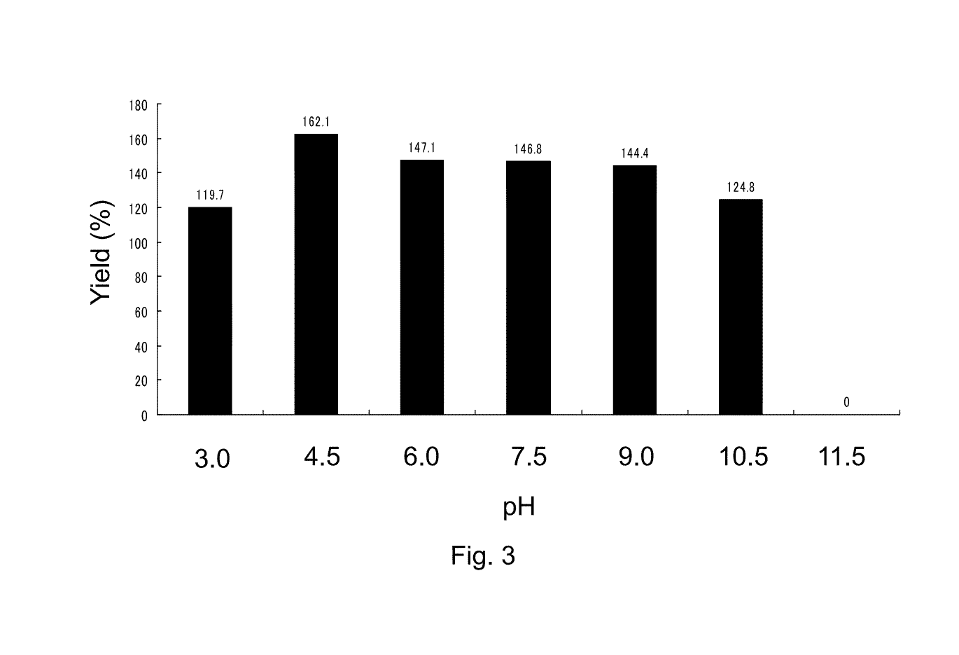

The culture fluid obtained in Example 1 was centrifuged, and sterilized water was added to the obtained precipitates to prepare a 1× suspension. A 1 N HCl solution or 1 N NaCl solution was added to the suspension to adjust the suspension to pH 3.0, pH 4.5, pH 6.0, pH 7.5, pH 9.0, pH 10.5, and pH 11.5, the suspensions were put into 1.5 ml-volume Eppendorf tubes in a volume of 1 ml each, and centrifuged again to separate the samples into supernatants with the various pH values and precipitates. The separated supernatant at each pH was added in a volume of 140 μL to the precipitates separated from the same sample, and 60 μL methanol was added to the mixture to obtain a suspension containing 30% methanol. The suspensions were incubated at 30° C. for 1 hour with rotation shaking at 1,000 rpm to allow the transesterification reaction. Then, lipids were extracted from the obtained samples with an organic solvent according to the Bligh-Dyer method, and fatty acid methyl esters were measured. The measurement results are shown in FIG. 3. When the reaction was allowed within an acidic region represented by pH 3.0 and an alkaline region of pH 10.5 or higher, the fatty acid ester yield tended to become low. In contrast, within a weakly acidic to weakly alkaline region of pH 4.5, pH 6.0, pH 7.5, and pH 9.0, high fatty acid ester yields were observed. In particular, the weakly acidic condition of pH 4.5 provided the highest fatty acid ester yield.

### Example 5

### Examination of Reaction Time for Reaction of Alga Culture after Addition of Alcohol

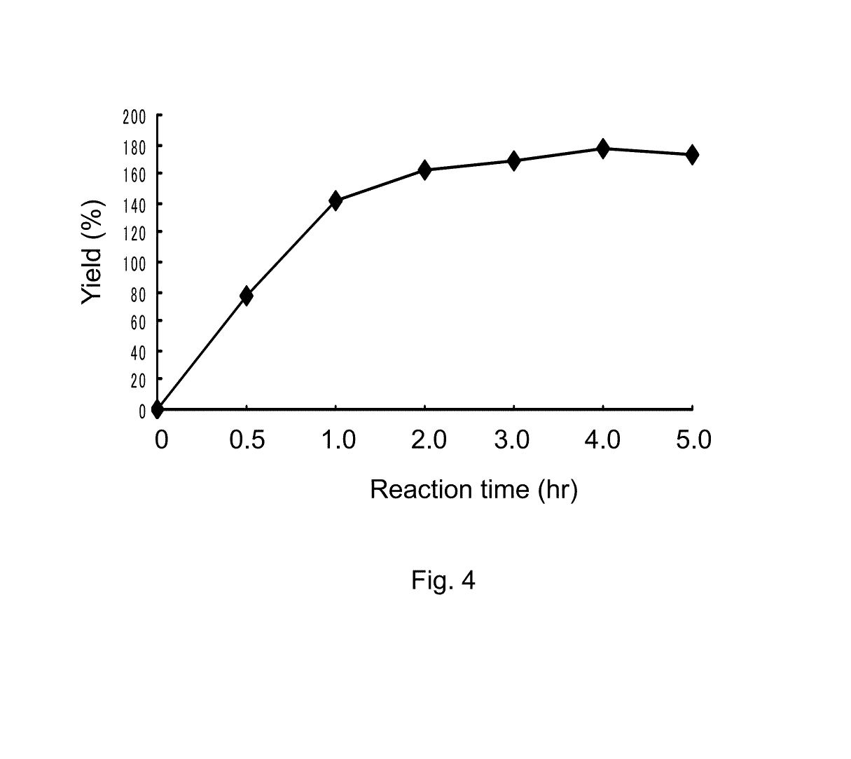

The culture fluid obtained in Example 1 was centrifuged, and sterilized water was added to the obtained precipitates to prepare a 1× suspension. The suspension was put into 1.5 ml-volume Eppendorf tubes in a volume of 1 ml each, and centrifuged again, and 200 μL each of a 30% aqueous solution of methanol was added to the obtained precipitates to obtain suspensions. The suspensions were incubated at 35° C. for 0.5, 1.0, 2.0, 3.0, 4.0, and 5.0 hours with rotation shaking at 1,000 rpm to allow the transesterification reaction. Then, lipids were extracted from the obtained samples with an organic solvent according to the Bligh-Dyer method, and fatty acid methyl esters were measured. The measurement results are shown in FIG. 4. With the lapse of the reaction time (0.5, 1.0 and 2.0 hours), the fatty acid methyl ester production yield increased, and after 2 hours, a substantially constant fatty acid methyl ester production yield was observed.

### Example 6

### Examination of Type of Alcohol to be Added for Reaction of Alga Culture after Addition of Alcohol

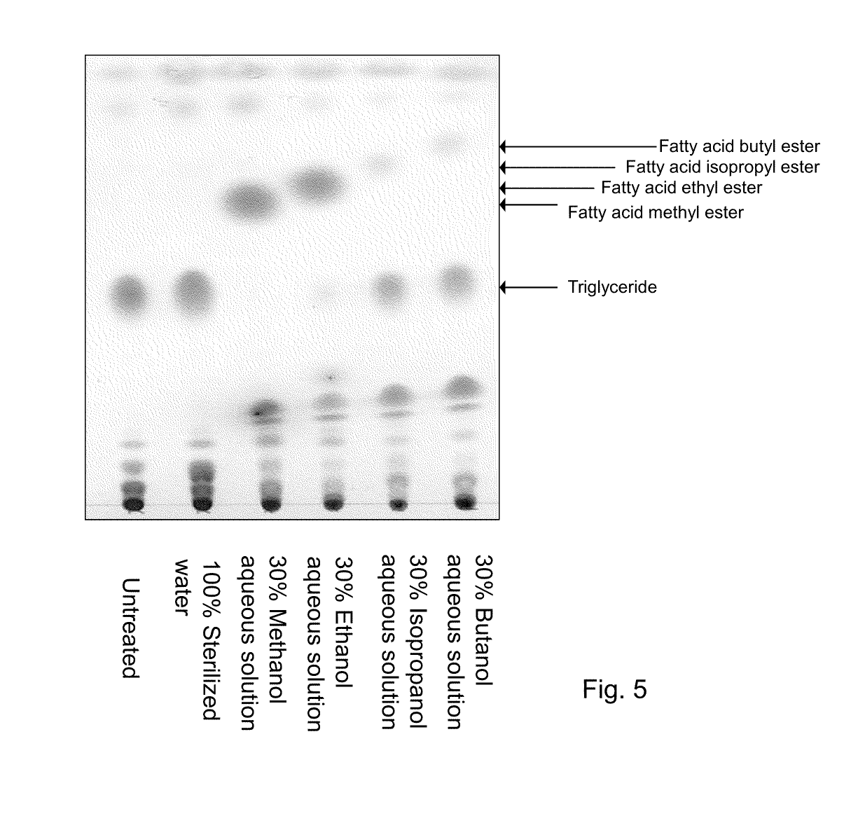

The culture fluid obtained in Example 1 was put into 1.5 ml-volume Eppendorf tubes in a volume of 1 ml each, and centrifuged, and 140 μL each of sterilized water was added to the obtained precipitates to prepare suspensions. To the suspensions, 60 μL each of sterilized water, methanol, ethanol, isopropanol, and butanol were added, so that the suspensions to which the alcohols were added became 30% alcohol solutions. The suspensions were stirred at 25° C. for 5 hours on a vortex mixer to allow the transesterification reaction. Then, lipids were extracted from the obtained samples with an organic solvent according to the Bligh-Dyer method, and types of fatty acid alcohol esters were identified by TLC. The test results are shown in FIG. 5. For the untreated sample and the sample to which 100% sterilized water was added, spots of triglycerides as the substrate of the transesterification reaction were confirmed, and spots of fatty acid alcohol esters were not confirmed. In contrast, for the samples as 30% ethanol solution, 30% isopropanol solution, and 30% butanol solution, spots of fatty acid ethyl esters, isopropyl esters, and butyl esters were confirmed, as in the case of the sample as 30% methanol solution.

### Example 7

### Culture of Microalga Strain, Chlorella kessleri 10 h, on 6-Well Plate

The Chlorella kessleri 11 h strain was cultured at 25° C., a light intensity of 7,000 luxes as a daylight condition (culture apparatus CL-301, TOMY), and a CO₂ concentration of 1% in the culture apparatus for 10 days in 5 mL of the 0.2× Gamborg's B5 medium (NIHON PHARMACEUTICAL) contained in wells of a 6-well plate, and the resultant culture fluid was used as a preculture. As the light source, white light from a fluorescent lamp was used, and as for the daylight condition, there was used a cycle in which the light intensity was increased from 0 lux to 7,000 luxes over 1 hour, maintained at 7,000 luxes for 11 hours, then decreased from 7,000 luxes to 0 lux over 1 hour, and maintained at 0 lux for 11 hours. The preculture in a volume of 0.1 mL was added to 5 mL of the 0.2× Gamborg's B5 medium (NIHON PHARMACEUTICAL) contained in wells of a 6-well plate, and culture was performed for 10 days under the same conditions.

0.2× Gamborg's B5 Medium:

| | | |
|---|---|---|
| KNO₃ | 500 | mg/L |
| MgSO₄•7H₂O | 50 | mg/L |
| NaH₂PO₄ •H₂O | 30 | mg/L |
| CaCl₂•2H₂O | 30 | mg/L |
| (NH₄)₂SO₄ | 26.8 | mg/L |
| Na₂-EDTA | 7.46 | mg/L |
| FeSO₄•7H₂O | 5.56 | mg/L |
| MnSO₄•H₂O | 2 | mg/L |
| H₃BO₃ | 0.6 | mg/L |
| ZnSO₄•7H₂O | 0.4 | mg/L |
| KI | 0.15 | mg/L |
| Na₂MoO₂•2H₂O | 0.05 | mg/L |
| CuSO₄•5H₂O | 0.005 | mg/L |
| CoCl₂•6H₂O | 0.005 | mg/L |

The medium was sterilized by autoclaving at 120° C. for 15 minutes.

### Example 8

### Identification of Fatty Acid Methyl Esters Produced in Reaction of Alga Culture after Addition of Alcohol

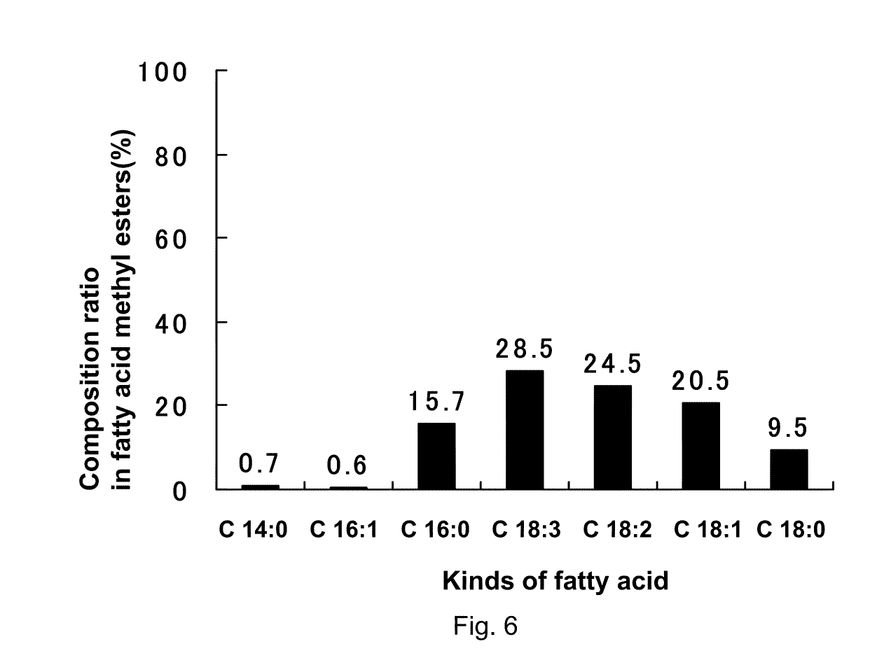

The culture fluid obtained in Example 7 was put into 1.5 ml-volume Eppendorf tube in a volume of 1 ml, and centrifuged, and 200 μL of a 30% aqueous solution of methanol was added to the obtained precipitates to prepare a suspension. The suspension was incubated at 30° C. for 4 hours with rotation shaking at 1,000 rpm to allow the transesterification reaction. Then, lipids were extracted from the obtained sample with an organic solvent according to the Bligh-Dyer method, and fatty acid methyl esters were identified. The test results are shown in FIG. 6. As the main components, α-linolenic acid methyl ester, linolic acid methyl ester, oleic acid methyl ester, palmitic acid methyl ester, and stearic acid methyl ester were confirmed. As other trace components, palmitoleic acid methyl ester and myristic acid methyl ester were confirmed.

### Example 9

### Culture of Chlorophyceae Algae

As Chlorophyceae algae, there were used Dunaliella tertiolecta UTEX LB999, Nannochloris oculata UTEX LB1998, Nannochloris sp. UTEX LB1999, Neochloris oleoabundans UTEX 1185, Scenedesmus obliquus UTEX B2630, Scenedesmus dimorphus UTEX417, Chlorella kessleri UTEX 398, Chlorella kessleri 11 h, and Chlorella sorokiniana UTEX 1230. The culture was performed by using the Daigo's IMK medium for marine microalgae for the following three strains, Dunaliella tertiolecta UTEX LB999, Nannochloris oculata UTEX LB1998, and Nannochloris sp. UTEX LB1999, the BG-11 medium for the following three strains, Scenedesmus obliquus UTEX B2630, Scenedesmus dimorphus UTEX417, and Chlorella sorokiniana UTEX 1230, the 0.2× Gamborg's medium for the following two strains, Chlorella kessleri UTEX 398 and Chlorella kessleri 11 h, and the Modified Bold 3N medium for Neochloris oleoabundans UTEX 1185 under the following conditions. Each Chlorophyceae alga was cultured at a light intensity of 7,000 luxes as a daylight condition (culture apparatus CL-301, TOMY), and a CO₂ concentration of 1% in the culture apparatus for 10 days in 5 mL of the corresponding medium contained in wells of a 6-well plate, and the resultant culture fluid was used as a preculture. As the light source, white light from a fluorescent lamp was used, and as for the daylight condition, there was used a cycle in which the light intensity was increased from 0 lux to 7,000 luxes over 1 hour, maintained at 7,000 luxes for 11 hours, then decreased from 7,000 luxes to 0 lux over 1 hour, and maintained at 0 lux for 11 hours. The preculture in a volume of 0.1 mL or 0.25 mL was added to 5 mL of the corresponding medium contained in wells of a 6-well plate, and culture was performed for 10 days under the same conditions.

Modified Bold 3N Medium:

| | | |
|---|---|---|
| NaNO₃ | 750 | mg/L |
| MgSO₄•7H₂O | 75 | mg/L |
| KH₂PO₄ | 175 | mg/L |
| K₂HPO₄ | 75 | mg/L |
| CaCl₂•2H₂O | 25 | mg/L |
| NaCl | 25 | mg/L |
| Na₂EDTA•2H₂O | 4.5 | mg/L |
| FeCl₃•6H₂O | 0.582 | mg/L |
| MnCl₂•4H₂O | 0.246 | mg/L |
| ZnCl₂ | 0.03 | mg/L |
| CoCl₂•6H₂O | 0.012 | mg/L |
| Na₂MoO₄•2H₂O | 0.024 | mg/L |
| HEPES | 0.036 | mg/L |
| Thiamine | 1.1 | mg/L |
| Biotin | 0.025 | mg/L |
| Vitamin B₁₂ | 0.12 | mg/L |
| CaCO₃ | 0.2 | mg/L |
| Green house soil | 0.2 | tsp/L |

The medium was adjusted to pH 6.2 and then sterilized by autoclaving at 120° C. for 15 minutes.

Daigo's IMK Medium for Marine Microalgae:

| | | |
|---|---|---|
| NaNO₃ | 200 | mg/L |
| Na₂HPO₄ | 1.4 | mg/L |
| K₂HPO₄ | 5 | mg/L |
| NH₄Cl | 2.68 | mg/L |
| Fe-EDTA | 5.2 | mg/L |
| Mn-EDTA | 0.332 | mg/L |
| Na₂-EDTA | 37.2 | mg/L |
| ZnSO₄•7H₂O | 0.023 | mg/L |
| CoSO₄•7H₂O | 0.014 | mg/L |
| Na₂MoO₄•2H₂O | 0.0073 | mg/L |
| CuSO₄•5H₂O | 0.0025 | mg/L |
| H₂SeO₃ | 0.0017 | mg/L |
| Thiamin-HCl | 0.2 | mg/L |
| Biotin | 0.0015 | mg/L |
| Vitamin B₁₂ | 0.0015 | mg/L |
| MnCl₂•4H₂O | 0.18 | mg/L |
| Daigo's artificial seawater SP | 36 | g/L |

The medium was adjusted to pH 8.0 with 1 N KOH, and then sterilized by autoclaving at 120° C. for 10 minutes.

0.2× Gamborg's B5 Medium:

| | | |
|---|---|---|
| KNO₃ | 500 | mg/L |
| MgSO₄•7H₂O | 50 | mg/L |
| NaH₂PO₄•H₂O | 30 | mg/L |
| CaCl₂•2H₂O | 30 | mg/L |
| (NH₄)₂SO₄ | 26.8 | mg/L |
| Na₂-EDTA | 7.46 | mg/L |
| FeSO₄•7H₂O | 5.56 | mg/L |
| MnSO₄•H₂O | 2 | mg/L |
| H₃BO₃ | 0.6 | mg/L |
| ZnSO₄•7H₂O | 0.4 | mg/L |
| KI | 0.15 | mg/L |
| Na₂MoO₂•2H₂O | 0.05 | mg/L |
| CuSO₄•5H₂O | 0.005 | mg/L |
| CoCl₂•6H₂O | 0.005 | mg/L |

The medium was sterilized by autoclaving at 120° C. for 15 minutes.

BG-11 Medium:

| | | |
|---|---|---|
| NaNO₃ | 1500 | mg/L |
| MgSO₄•7H₂O | 7.5 | mg/L |
| K₂HPO₄ | 40 | mg/L |
| CaCl₂•2H₂O | 36 | mg/L |
| Citric acid•H₂O | 6 | mg/L |
| Na₂EDTA•2H₂O | 1 | mg/L |
| Ferric ammonium citrate | 6 | mg/L |
| Na₂CO₃ | 20 | mg/L |
| MnCl₂•4H₂O | 1.81 | mg/L |
| ZnSO₄•7H₂O | 0.22 | mg/L |
| Co(NO₃)₂•6H₂O | 0.0494 | mg/L |
| Na₂MoO₄•2H₂O | 0.39 | mg/L |
| CuSO₄•5H₂O | 0.079 | mg/L |
| H₃BO₃ | 2.86 | mg/L |

The medium was adjusted to pH 7.2 with 1 N KOH, and then sterilized by autoclaving at 120° C. for 10 minutes.

### Example 10

### Implementation of Reaction of Chlorophyceae Alga Culture after Addition of Alcohol

Each culture fluid obtained in Example 9 in a volume of 1 ml was put into 1.5 ml-volume Eppendorf tube, and centrifuged, and 200 μL of a 30% aqueous solution of methanol was added to the obtained precipitates to prepare a suspension. The suspension was incubated at 30° C. for 4 hours with rotation shaking at 1,000 rpm to allow the transesterification reaction. Then, lipids were extracted from the obtained sample with an organic solvent according to the Bligh-Dyer method, and fatty acid methyl esters were identified. The test results are shown in

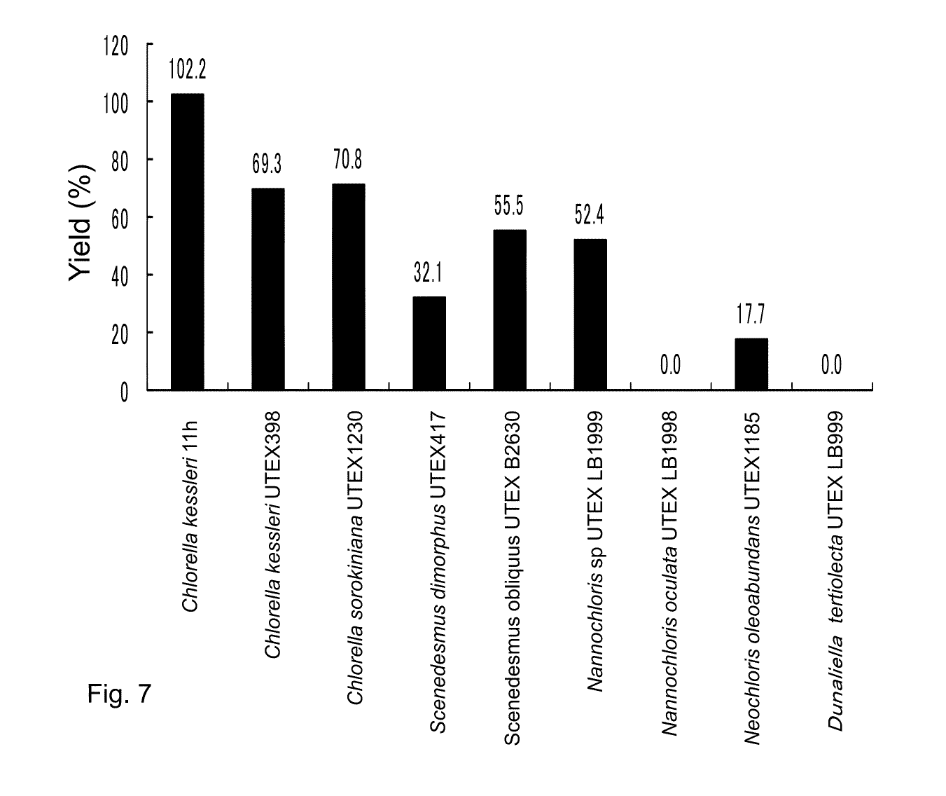

FIG. 7. With the freshwater Chlorophyceae algae of the genus Chlorella or Scenedesmus, there was observed a tendency that a relatively high fatty acid methyl ester production yield was obtained. In contrast, as for the marine Chlorophyceae algae of the genus Nannochloris, Neochloris, or Dunaliella, a relatively high fatty acid methyl ester production yield was obtained with Nannochloris sp. UTEX1999, but no fatty acid methyl ester production was observed at all with Nannochloris oculata UTEX1998 of the same genus or one strain of the genus Dunaliella.

### Example 11

### Confirmation of Fatty Acid Production in Reaction of Alga Culture after Addition of Organic Solvent Other than Alcohol

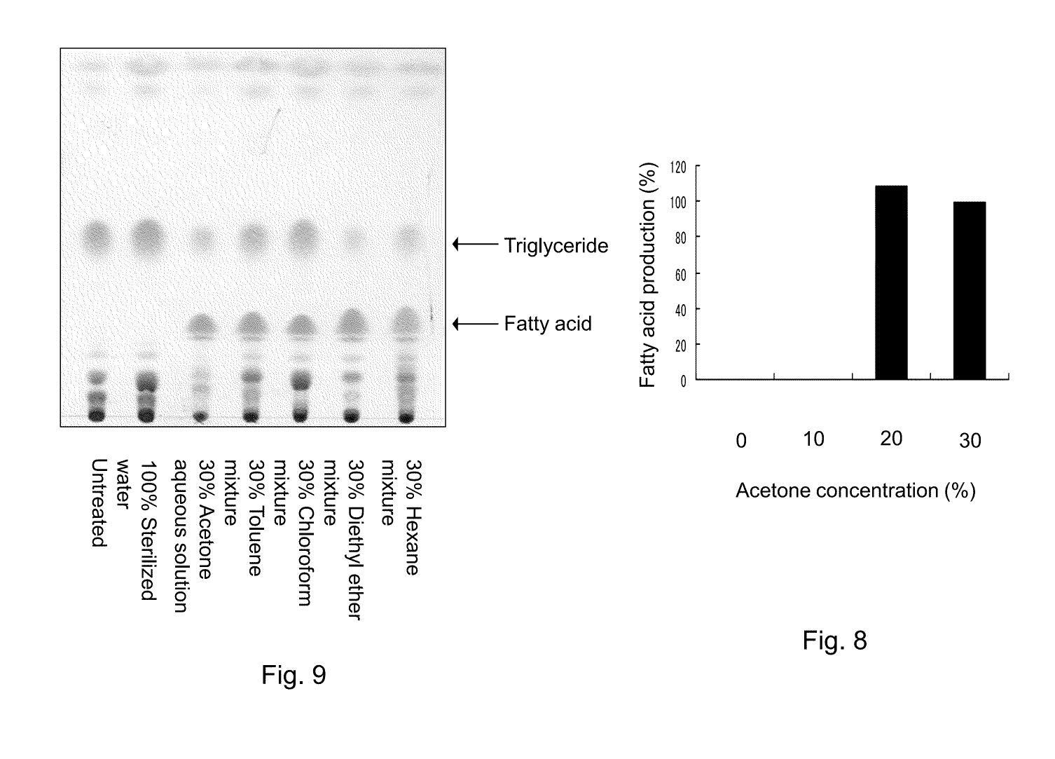

The culture fluid obtained in Example 1 was put into 1.5 ml-volume Eppendorf tubes in a volume of 1 ml each, and centrifuged, and 200 μl each of aqueous solutions of acetone at acetone concentrations of 10%, 20%, and 30% were added to the obtained precipitates to prepare suspensions. The suspensions were stirred at 25° C. for 5 hours on a vortex mixer to allow hydrolysis of lipids. Then, lipids were extracted from the obtained samples with an organic solvent according to the Bligh-Dyer method, and types of fatty acids were identified by TLC. The test results are shown in FIG. 8. With the 10% aceton solution, no fatty acid production was confirmed at all, but with aceton solutions at concentrations of 10% and 20%, high fatty acid production yields were confirmed.

### Example 12

### Examination of Kind of Organic Solvent to be Added for Fatty Acid Production in Reaction of Alga Culture after Addition of Organic Solvent

The culture fluid obtained in Example 1 was put into 1.5 ml-volume Eppendorf tubes in a volume of 1 ml each, and centrifuged, and 140 μL each of sterilized water was added to the obtained precipitates to prepare suspensions. To the suspensions, 60 μL each of sterilized water, acetone, toluene, chloroform, diethyl ether, and hexane were added, so that the suspensions to which the organic solvents were added became 30% organic solvent solutions or mixtures. The suspensions were stirred at 25° C. for 5 hours on a vortex mixer to allow hydrolysis of lipids. Then, lipids were extracted from the obtained samples with an organic solvent according to the Bligh-Dyer method, and types of fatty acids were identified by TLC. The results are shown in FIG. 9. With the untreated sample and the sample to which 100% sterilized water was added, spots of triglycerides as the substrate of the hydrolysis were confirmed, and spots of fatty acids were not confirmed. In contrast, with the samples as 30% toluene mixture, 30% chloroform mixture, 30% diethyl ether mixture, and 30% hexane mixture, decrease of spots of triglycerides, and emergence of spots of fatty acids were also confirmed, as in the case of using the sample as 30% acetone solution.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

1. A method for producing a fatty acid, which comprises:
(a) culturing an alga in a culture medium;
(b) adding an organic solvent to the culture medium to obtain a mixture,
(c) stirring the mixture to allow a transesterification or hydrolysis reaction of a lipid, and
(d) collecting a fatty acid ester or a fatty acid from the mixture.

2. The method according to claim 1, wherein the organic solvent is ethanol, and a fatty acid ester is collected.

3. The method according to claim 1, wherein the organic solvent is selected from the group consisting of acetone, chloroform, ethyl acetate, methyl acetate, hexane, benzene, toluene, dichloromethane, acetonitrile, dimethyl ether, and diethyl ether; and a fatty acid is collected.

4. The method according to claim 1, wherein concentration of the organic solvent in the mixture is 5% or higher.

5. The method according to claim 1, wherein concentration of the organic solvent in the mixture is 65% or lower.

6. The method according to claim 1, wherein the organic solvent is a lower alcohol having 5 or less carbon atoms.

7. The method according to claim 1, wherein the organic solvent is a higher alcohol having 6 or more carbon atoms.

8. The method according to claim 1, wherein the reaction is allowed at a temperature of 10° C. or higher.

9. The method according to claim 1, wherein the reaction is allowed at a temperature of 60° C. or lower.

10. The method according to claim 1, wherein the reaction is allowed at a weakly acidic to weakly alkaline pH.

11. The method according to claim 1, wherein the collecting the fatty acid ester or fatty acid comprises treating the reaction product with an organic solvent.

12. The method according to claim 1, wherein the alga is a microalga.

13. The method according to claim 12, wherein the microalga belongs to the phylum Chlorophyta.

14. The method according to claim 13, wherein the microalga is a microalga belonging to the class Chlorophyceae, Trebouxiophyceae, or Prasinophyceae.

15. The method according to claim 14, wherein the microalga is a microalga belonging to the class Chlorophyceae.

16. The method according to claim 12, wherein the alga is a freshwater microalga belonging to the class Chlorophyceae.

17. The method according to claim 12, wherein the alga is a marine microalga belonging to the class Chlorophyceae, and is a microalga that accumulates fats and oils as storage substances.

18. A method for producing an L-amino acid, which comprises:
(a) producing a fatty acid by the method according to claim 1
(b) culturing a bacterium having an L-amino acid-producing ability in a medium containing the fatty acid produced in (a) to produce and accumulate an L-amino acid in culture, and
(c) collecting the L-amino acid from the culture.

19. The method according to claim 18, wherein the bacterium is a bacterium belonging to the family Enterobacteriaceae or a coryneform bacterium.

20. The method according to claim 19, wherein the bacterium belonging to the family Enterobacteriaceae is Escherichia coli.
